SEQUENCE LISTING (36 sequences)

SEQ ID NO: 1 - DNA, 20 bp, Artificial Sequence
  Tag
caagatgctacgcttcagtc

SEQ ID NO: 2 - DNA, 43 bp, Artificial Sequence
  Primer for rs13182993
caagatgctacgcttcagtcaggagactatgaggtgtgtctct

SEQ ID NO: 3 - DNA, 39 bp, Artificial Sequence
  Primer for rs13182993
caagatgctacgcttcagtccctgtgcacctcgattgaa

SEQ ID NO: 4 - DNA, 39 bp, Artificial Sequence
  Primer for rs560681
caagatgctacgcttcagtcccaaggggaatcacacctc

SEQ ID NO: 5 - DNA, 40 bp, Artificial Sequence
  Primer for rs560681
caagatgctacgcttcagtctctgtggaagcatgccactc

SEQ ID NO: 6 - DNA, 40 bp, Artificial Sequence
  Primer for rs740598
caagatgctacgcttcagtctgctgagccactctttcagg

SEQ ID NO: 7 - DNA, 40 bp, Artificial Sequence
  Primer for rs740598
caagatgctacgcttcagtcttccgggatgtcccgtctta

SEQ ID NO: 8 - DNA, 43 bp, Artificial Sequence
  Primer for rs1358856
caagatgctacgcttcagtcacaggcaaagaggaacatacagt

SEQ ID NO: 9 - DNA, 44 bp, Artificial Sequence
  Primer for rs1358856
caagatgctacgcttcagtctgctggcagtgttatttctttctc

SEQ ID NO: 10 - DNA, 37 bp, Artificial Sequence
  Primer for rs9951171
caagatgctacgcttcagtcctcgttgttcctctggg

SEQ ID NO: 11 - DNA, 39 bp, Artificial Sequence
  Primer for rs9951171
caagatgctacgcttcagtcctgttcaagggaagcctgt

SEQ ID NO: 12 - DNA, 40 bp, Artificial Sequence
  Primer for rs7520386
caagatgctacgcttcagtcggatcaggaaacagggagcc

SEQ ID NO: 13 - DNA, 40 bp, Artificial Sequence
  Primer for rs7520386
caagatgctacgcttcagtcgaagactctgtcccagccac

SEQ ID NO: 14 - DNA, 40 bp, Artificial Sequence
  Primer for rs13218440
caagatgctacgcttcagtcgcttcttctgccacatccct

SEQ ID NO: 15 - DNA, 40 bp, Artificial Sequence
  Primer for rs13218440
caagatgctacgcttcagtcgcattttcatggagggccac

SEQ ID NO: 16 - DNA, 40 bp, Artificial Sequence
  Primer for rs279844
caagatgctacgcttcagtcttgccatgtttgtcacaggt

SEQ ID NO: 17 - DNA, 45 bp, Artificial Sequence
  Primer for rs279844
caagatgctacgcttcagtcaccttggtttcttgattatgttgat

SEQ ID NO: 18 - DNA, 39 bp, Artificial Sequence
  Primer for rs1058083
caagatgctacgcttcagtctgaatcctcccccaagctg

SEQ ID NO: 19 - DNA, 40 bp, Artificial Sequence
  Primer for rs1058083
caagatgctacgcttcagtcttctcctcttcctgggctga

SEQ ID NO: 20 - DNA, 40 bp, Artificial Sequence
  Primer for rs2032597
caagatgctacgcttcagtcgcacattaaatgggttccag

SEQ ID NO: 21 - DNA, 44 bp, Artificial Sequence
  Primer for rs2032597
caagatgctacgcttcagtcgaaatacgaaggacacaaaacctc

SEQ ID NO: 22 - DNA, 40 bp, Artificial Sequence
  Primer for rs2032631
caagatgctacgcttcagtctgttgctggcaagacacttc

SEQ ID NO: 23 - DNA, 41 bp, Artificial Sequence
  Primer for rs2032631
caagatgctacgcttcagtctgcctttgctacaactctcct

SEQ ID NO: 24 - DNA, 39 bp, Artificial Sequence
  Primer for rs2272998
caagatgctacgcttcagtctagccccacgtcacttcag

SEQ ID NO: 25 - DNA, 40 bp, Artificial Sequence
  Primer for rs2272998
caagatgctacgcttcagtcttcttggaaggtggtcctgg

SEQ ID NO: 26 - DNA, 43 bp, Artificial Sequence
  Primer for rs12997453
caagatgctacgcttcagtcaggacctgtaagagtctgtgatt

SEQ ID NO: 27 - DNA, 43 bp, Artificial Sequence
  Primer for rs12997453
caagatgctacgcttcagtctgtatcccaggttcaatgactgt

SEQ ID NO: 28 - DNA, 43 bp, Artificial Sequence
  Primer for rs214955
caagatgctacgcttcagtcacacccttacctgtattttctga

SEQ ID NO: 29 - DNA, 44 bp, Artificial Sequence
  Primer for rs214955
caagatgctacgcttcagtcgtgcacattctaagaactggtgat

SEQ ID NO: 30 - DNA, 41 bp, Artificial Sequence
  Primer for rs13134862
caagatgctacgcttcagtctgcttacagtgattcttgcct

SEQ ID NO: 31 - DNA, 43 bp, Artificial Sequence
  Primer for rs13134862
caagatgctacgcttcagtcagtcttttgcaccaagtcttttt

SEQ ID NO: 32 - DNA, 41 bp, Artificial Sequence
  Primer for rs1410059
caagatgctacgcttcagtcggaagatgcttgaactcccca

SEQ ID NO: 33 - DNA, 39 bp, Artificial Sequence
  Primer for rs1410059
caagatgctacgcttcagtcacatcaaagctgggaaccg

SEQ ID NO: 34 - protein, 62 aa, Homo sapiens
MKFLVFAFILALMVSMIGADSSEEKFLRRIGRFGYGYGPYQPVPEQPLYPQPYQPQYQQYTF

SEQ ID NO: 35 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 36 - protein, 148 aa, Homo sapiens
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

(19) United States
(12) Patent Application Publication
(10) Pub. No.: US 2018/0328912 A1
(43) Pub. Date: Nov. 15, 2018

(54) METHODS OF MATCHING A URINE SAMPLE TO A SUBJECT AND CLINICAL USES THEREOF

(71) Applicant: Genotox ID LLC, Austin, TX (US)

(72) Inventor: Matt McCarty, Austin, TX (US)

(73) Assignee: Genotox ID LLC, Austin, TX (US)

(21) Appl. No.: 15/875,399

(22) Filed: Jan. 19, 2018

Related U.S. Application Data
(60) Provisional application No. 62/448,201, filed on Jan. 19, 2017.

(51) Int. Cl. G01N 33/493; C12Q 1/6876

(52) U.S. Cl. CPC G01N 33/493; C12Q 1/6876; C12Q 2600/156; A61B 5/117; C12Q 2600/136; C12Q 2600/166; C12Q 2600/16

(57) ABSTRACT

Provided herein are methods of matching a urine sample to a subject that include enriching a urine sample for mammalian cells, isolating any genomic DNA from the enriched sample to form an isolated genomic DNA sample, contacting a set of two or more oligonucleotide probes to the genomic DNA in the isolated genomic DNA test sample, where each of the two or more oligonucleotide probes hybridizes to a sequence of genomic DNA that is one nucleotide upstream of a different target single nucleotide polymorphism (SNP), elongating the two or more oligonucleotide probes to generate a set of two or more SNP amplification products; determining the mass distribution of the set of two or more SNP amplification products; and comparing the determined mass distribution of the set of SNP products to a control mass distribution.

### CROSS-REFERENCE TO RELATED APPLICATION

This application claims priority to U.S. Provisional Patent Application Ser. No. 62/448,201, filed Jan. 19, 2017, which is herein incorporated by reference in its entirety.

### TECHNICAL FIELD

This invention relates to methods of molecular biology and urine testing.

### BACKGROUND

Urine drug testing is a commonly used tool to detect a subject's use of drugs, both legal (e.g., controlled substances) and illegal. During the last half century, the use of urine drug testing has been used throughout the military, in the public and private workplace, in courts, in professional sports, and in medical clinics and care centers. The urine drug tests are used primarily to detect illegal or banned substances in a subject's system. In the clinical setting, physicians test their patients to determine if their patients are adhering to their prescriptions. Urine drug testing has become a routinely used effective tool in the assessment and ongoing management of patients who are treated with controlled substances for, e.g., chronic pain. The urine drug testing results provide confirmation of the agreed-upon treatment plan and diagnose relapse or drug abuse.

The results of a urine drug test can have serious consequences for a patient including termination of prescription. In fear of the possible consequences, patients have developed a variety of methods to cheat by substituting their own urine sample with that of others. Patients who “cheat” a urine drug test by using adulterated samples (e.g., another person's urine) or synthetic urine present a problem for the treating medical doctor because the ongoing care plan will not be based on accurate information. Currently, the best method for validating that a patient's sample is in fact their own is by observation during sample collection (e.g., a witnessed urine test)—which is not always possible. Another complication of urine drug testing is that a clinical lab can mix-up urine samples, which also leads to inaccurate test results.

### SUMMARY

The present invention is based on the development of methods of matching a urine sample to a subject that include, in part: (a) enriching a urine sample from a subject for mammalian cells (if present), (b) isolating any genomic DNA from the enriched sample of step (a); (c) contacting a set of two or more oligonucleotide probes to the genomic DNA in the isolated genomic DNA test sample, where each of the two or more oligonucleotide probes hybridizes to a sequence of genomic DNA that is one nucleotide upstream of a different target single nucleotide polymorphism (SNP); (d) elongating the two or more oligonucleotide probes hybridized to the genomic DNA by one nucleotide using chain-termination nucleotide(s) to generate a set of two or more SNP amplification products; (e) determining the mass distribution of the set of SNP amplification products; and (f) comparing the determined mass distribution of the set of SNP amplification products to a control mass distribution.

Provided herein are methods of matching a urine sample to a subject that include: (a) providing a urine sample from a subject; (b) enriching the urine sample for mammalian cells, if present; (c) isolating any genomic DNA from the enriched sample of step (b) to form an isolated genomic DNA test sample; (d) contacting a set of two or more oligonucleotide probes to the genomic DNA in the isolated genomic DNA test sample, where each of the two or more oligonucleotide probes hybridizes to a sequence of genomic DNA that is one nucleotide upstream of a different target single nucleotide polymorphism (SNP); (e) elongating the two or more oligonucleotide probes hybridized to the genomic DNA by one nucleotide using chain-terminating nucleotide(s) to generate a set of two or more SNP amplification products; (f) determining the mass distribution of the set of SNP amplification products; (g) comparing the determined mass distribution of the set of SNP amplification products to a control mass distribution; and (h) identifying a urine sample having a mass distribution that is the same as the control mass distribution as originating from the subject; or identifying a urine sample having a mass distribution that is not the same as the control mass distribution as not originating from the subject.

In some embodiments of any of the methods described herein, the chain-terminating nucleotide(s) is/are dideoxynucleotide(s). In some embodiments of any of the methods described herein, the mass distribution of the set of SNP amplification products in step (f) is determined using matrix-assisted laser desorption/ionization time-of-flight (MALDI-TOF) analysis. In some embodiments of any of the methods described herein, each of the two or more oligonucleotide probes hybridizes to a sequence of genomic DNA that is one nucleotide upstream of a target SNP having a minor allele frequency of >0.4.

In some embodiments of any of the methods described herein, the set of oligonucleotide probes includes at least four (e.g., at least six, at least eight, at least ten, at least twelve, or at least fourteen) oligonucleotide probes. In some embodiments of any of the methods described herein, the target SNPs are selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments of any of the methods described herein, the target SNPs are selected from the group consisting of: rs7520386, rs560681, rs9951171, rs1058083, rs1358856, rs214955, rs740598, rs279844, rs13218440, rs2272998, rs12997453, rs13134862, rs13182883, and rs1410059.

In some embodiments of any of the methods described herein, the subject is a genetic male and at least one target SNP is located on a Y chromosome. In some embodiments of any of the methods described herein, the target SNPs include at least one target SNP from at least three different chromosomes. In some embodiments of any of the methods described herein, the target SNPs include at least one target SNP from at least eight different chromosomes.

Some embodiments of any of the methods described herein further include, between steps (c) and (d), a step of: performing a pre-amplification step. In some embodiments of any of the methods described herein, the pre-amplification step includes: hybridization of two or more pairs of a pre-amplification forward and reverse primer, where each pair of pre-amplification forward and reverse primers is designed to amplify 250 to 300 nucleotides of genomic DNA that contains one of the different target SNPs, where the pre-amplification forward and reverse primer in each of the pairs of pre-amplification primers contains (i) a sequence of about 17 to 25 contiguous nucleotides that is complementary to a sequence in the genomic DNA and (ii) a tag sequence of about 17 to about 25 contiguous nucleotides that is not complementary to a sequence in the genomic DNA; and amplification of the genomic DNA using the two or more pairs of pre-amplification forward and reverse primers to generate 250 to 300 nucleotide amplification product(s). In some embodiments of any of the methods described herein, the pre-amplification step further includes amplification of the 250 to 300 nucleotide amplification product(s) using a primer that includes a sequence of about 17 to 25 contiguous nucleotides complementary to the tag sequence.

In some embodiments of any of the methods described herein, the control mass distribution is determined from a buccal cell sample obtained from the subject. Some embodiments of any of the methods described herein further include obtaining the buccal cell sample from the subject. Some embodiments of any of the methods described herein further include determining the mass distribution of the buccal cell sample obtained from the subject. In some embodiments of any of the methods described herein, the subject is a human.

Some embodiments of any of the methods described herein further include: (i) performing an assay to identify the presence of one or more of statherin, alpha-amylase, and lysozyme in the urine sample; and (j) identifying a urine sample having a mass distribution that is the same as the control mass distribution and a detectable level of one or more of statherin, alpha-amylase, and lysozyme as being adulterated. In some embodiments of any of the methods described herein, the assay in step (i) is an enzyme activity assay. In some embodiments of any of the methods described herein, the assay in step (i) is an enzyme-linked immunosorbent assay (ELISA).

Some embodiments of any of the methods described herein further include recording the identification in step (h) in the subject's medical record (e.g., a computer readable medium).

Some embodiments of any of the methods described herein further include notifying the subject's insurance provider, employer, or potential future employer of the identification in step (h). Some embodiments of any of the methods described herein further include notifying a pharmacist or a medical professional of the identification in step (h).

Some embodiments of any of the methods described herein further include: (i) selecting a subject having a urine sample identified in step (h) as not originating from the subject; and (j) obtaining an additional urine sample from the selected subject. In some embodiments of any of the methods described herein, the additional urine sample is obtained through a witnessed urine test. Some embodiments of any of the methods described herein further include (k) performing an assay to determine the level of one or more drug metabolites in the additional urine sample. Some embodiments of any of the methods described herein further include: (l) identifying a subject having an elevated level of one or more drug metabolites in the additional urine sample as compared to a reference level of the one or more drug metabolites, where the drug metabolites are metabolites of an illegal or controlled substance; and (m) admitting the subject into the drug dependency program, ceasing administration of the controlled substance to the subject or reducing the dose and/or frequency of administration of the controlled substance to the subject. In some embodiments of any of the methods described herein, the drug dependency program includes administering to the subject in step (m) a drug replacement therapy.

Some embodiments of any of the methods described herein further include: (i) selecting a subject having a urine sample identified in step (h) as not originating from the subject; (j) obtaining a sample including blood, serum, hair, or plasma from the subject; and (k) performing an assay to determine the level of one or more drug metabolites in the sample from step (j). Some embodiments of any of the methods described herein further include: (l) identifying a subject having an elevated level of one or more drug metabolites in the sample from step (j) as compared to a reference level of the one or more drug metabolites in the sample from step (j) as compared to a reference level of the one or more drug metabolites, wherein the drug metabolites are metabolites of an illegal or controlled substance; and (m) admitting the subject into a drug dependency program, ceasing administration of the controlled substance to the subject, or reducing the dose or frequency of administration of the controlled substance to the subject. In some embodiments of any of the methods described herein, the drug dependency program includes administering to the subject in step (m) a drug replacement therapy.

In some embodiments of any of the methods described herein, the urine sample is identified in step (h) as originating from the subject. Some embodiments of any of the methods described herein further include performing an assay to determine the level of one or more drug metabolites in the urine sample identified in step (h) as originating from the subject. Some embodiments of any of the methods described herein further include: identifying a subject having an elevated level of one or more drug metabolites in the urine sample identified in step (h) as originating from the subject, as compared to a reference level of the one or more drug metabolites, wherein the drug metabolites are metabolites of an illegal or controlled substance; and admitting the subject into the drug dependency program, ceasing administration of the controlled substance to the subject or reducing the dose and/or frequency of administration of the controlled substance to the subject. In some embodiments of any of the methods described herein, the drug dependency program includes administering to the subject a drug replacement therapy.

In some embodiments of any of the methods described herein, the subject has not been diagnosed as having an illegal or controlled substance addiction. In some embodiments of any of the methods described herein, the subject has been identified as having an illegal or controlled substance addiction. In some embodiments of any of the methods described herein, the subject is being treated on an outpatient basis for an illegal or controlled substance addiction. In some embodiments of any of the methods described herein, the subject is entering into or participating in a medication monitoring program involving a controlled substance.

As used herein, the word “a” before a noun represents one or more of the particular noun. For example, the phrase “a SNP” represents “one or more SNPs.”

The term “subject” means a vertebrate, including any member of the class Mammalia, including humans, sports or pet animals, such as horse (e.g., race horse) or dog (e.g., race dogs), and higher primates. In preferred embodiments, the subject is a human.

The phrase “originating from the subject” when used to describe material or sample means a material (e.g., a biological fluid, e.g., urine) or sample (e.g., a sample of biological fluid, e.g., a urine sample) produced by the subject's own body and not including (even in part) a material (e.g., a biological fluid, e.g., urine) or sample (e.g., a sample of biological fluid, e.g., a urine sample) produced by another subject's body.

The phrase “not originating from the subject” when used to describe a material or sample means a material (e.g., a biological fluid, e.g., urine) or sample (e.g., a sample of biological fluid, e.g., a urine sample) that includes (at least in part) a material (e.g., a biological fluid, e.g., urine) or sample (e.g., a sample of biological fluid, e.g., a urine sample) produced by a different subject's body.

The term “adulterated sample” means a urine sample from a subject that has been manipulated to add genomic DNA from another subject's or the subject's body, where the added genomic DNA is from a source other than mammalian cells present in urine. Non-limiting examples of another subject include a friend, a spouse, or a non-human mammal (e.g., a domesticated animal). In some embodiments, the source of the added genomic DNA can be, e.g., blood, hair, eyelashes, skin cells, saliva, semen, tears, mucus (e.g., eye or nose mucus), phlegm, and/or buccal cells.

The term “synthetic urine” is art known and means a synthetic liquid that is not produced by the body of a mammal (e.g., human) that is meant to substitute for urine produced by the body of a mammal (e.g., a human). As is known in the art, synthetic urine is commercially available from a number of vendors.

The phrase “enriching a urine sample for mammalian cells, if present” means handling or processing a sample of urine in order to concentrate any mammalian cells, if present, in the sample. Non-limiting methods for enriching a urine sample for mammalian cells, if present, can include one or more steps of centrifugation (e.g., high speed centrifugation), beads coated with an antibody that specifically binds to an antigen present on the surface of mammalian cells, filtration, gravitational settling of the sample, and aspiration or removal of a supernatant substantially free of mammalian cells.

The term “mass distribution” is an art known term and means a distribution or representation of the relative abundance of multiple different chemical species present in a composition, where at least two or more of the multiple different chemical species each has a different (e.g., detectably different) mass. For example, a mass distribution can refer to the collection of intensity values of spectra obtained for a set of SNP amplification products using matrix-assisted laser desorption/ionization time-of-flight (MALDI-TOF). Additional examples of mass distributions and methods of determining a mass distribution are described herein. Additional examples of mass distributions and methods of determining a mass distribution are known in the art.

The term “chain-terminating nucleotide” means a nucleotide that can be covalently linked to the 3′-OH end of a pre-existing nucleotide (e.g., the 3′-OH terminal end of an oligonucleotide, e.g., a primer) via the activity of a DNA and/or RNA polymerase, but itself does not have a 3′-OH end that can thereafter be used by a DNA and/or RNA polymerase to covalently link it to another nucleotide. Non-limiting examples of chain-terminating nucleotides are described herein. Additional examples of chain-terminating nucleotides are known in the art.

The term “drug metabolite” is art known and means a break-down product of a controlled or illegal substance produced by a mammal's body following administration of the controlled or illegal substance to the mammal (e.g., human). A wide variety of drugs, drug metabolites, and assays for detecting the levels of drugs and drug metabolites are known in the art. Non-limiting examples of drugs, drug metabolites, and vendors that sell kits for determining the level of one or more drugs and drug metabolites are described herein.

The term “controlled substance” means an agent or material that is regulated by a government (e.g., state government, federal government, and/or a governmental drug regulatory agency, such as the U.S. Food and Drug Administration), but its administration to at least some persons is not illegal. For example, the dosage and frequency of administration of a controlled substance can be regulated by a government. In some examples, certain persons in a population are warned not to consume a controlled substance. Non-limiting examples of controlled substances are prescription drugs and marijuana.

The term “addiction” means a chronic disease characterized by the compulsive behavior and inability of a subject to voluntarily control, stop, or withdraw from using an illegal or a controlled substance. Additional aspects of addiction are known in the art. The term “drug replacement therapy” means administration of an agent that mimics the pharmacological effect of a controlled or illegal substance but is longer acting, less potent, less toxic, and/or has an improved safety profile than the controlled or illegal substance. Exemplary aspects of drug replacement therapies are described herein. Additional aspects of drug replacement therapies are known in the art.

The term “potential future employer” means a person or business entity that is considering a subject for employment and that requires or asks employment candidates to provide a urine sample for testing as part of the job application process. For example, a potential future employer can be a state or federal government, a medical care facility (e.g., a clinic or a hospital), a transportation company, a professional sports team, or an airline company.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description, and from the claims.

### BRIEF DESCRIPTION OF THE FIGURES

FIG. 1 shows the results of DNA authentication, synthetic urine screening, and validity performed as described in Example 1.

FIG. 2 shows that prior urine validity tests (not the methods described herein) failed to detect urine substitution 98% of the time.

FIG. 3 shows that the substitution of urine as detected using the methods described in Example 1 occurs about equally in male and female patients.

FIG. 4 shows the substitution of urine as detected using the methods described in Example 1 is most prevalent in persons aged 35 to 44, and 45 to 54.

FIG. 5 shows the relative likelihood of urine substitution as compared to an average patient, regardless of sex, as determined using the methods described in Example 1. Individuals aged 35 to 44 are 53% more likely to substitute than the average patient, regardless of sex.

FIG. 6 shows that the use of synthetic urine occurs with males slightly more than females, as determined using the methods of Example 1.

FIG. 7 shows that synthetic urine is used across all age groups as determined using the methods described in Example 1. The data show that patients aged 35 to 44 and 45 to 54 comprise the majority of persons engaged in the use of synthetic urine in a clinical setting. This result is similar to the use of human urine substitution, as determined by DNA authentication.

FIG. 8 shows the relative likelihood of the use of synthetic urine as compared to an average patient in a clinical setting, as determined using the methods described in Example 1. The data show that patients that are 21 and under or 45 to 54 show disproportionate use of synthetic urine compared to an average person in a clinical setting.

### DETAILED DESCRIPTION

Provided herein are methods of matching a urine sample to a subject that include: (a) providing a urine sample from a subject; (b) enriching the urine sample for mammalian cells, if present; (c) isolating any genomic DNA from the enriched sample of step (b) to form an isolated genomic DNA test sample; (d) contacting a set of two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more) oligonucleotide probes to the genomic DNA in the isolated genomic DNA test sample, where each of the two or more oligonucleotide probes hybridizes to a sequence of genomic DNA that is one nucleotide upstream of a different target single nucleotide polymorphism (SNP); (e) elongating the two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more) oligonucleotide probes hybridized to the genomic DNA by one nucleotide using chain-terminating nucleotide(s) to generate a set of two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, or forty or more) SNP amplification products; (f) determining the mass distribution of the set of amplification products; (g) comparing the determined mass distribution of the set of SNP amplification products to a control mass distribution; and (h) identifying a urine sample having a mass distribution that is the same as the control mass distribution as originating from the subject; or identifying a urine sample having a mass distribution that is not the same as the control mass distribution as not originating from the subject. Also provided are clinical uses of these methods of matching a urine sample to a subject.

Non-limiting aspects of these methods for matching a urine sample to a subject and the clinical uses of these methods are described herein and can be used in any combination.

### Subjects

In any of the methods described herein, the subject has not been diagnosed as having an illegal or controlled substance addiction. In some embodiments of any of the methods described herein, the subject has been identified as having an illegal or controlled substance addiction (e.g., a subject that has already undergone treatment (e.g., successful or unsuccessful treatment) for his or her illegal or controlled substance addiction). In some embodiments of any of the methods described herein, the subject is being treated on an outpatient basis for an illegal or controlled substance addiction. In some embodiments, the subject is receiving inpatient treatment for his or her illegal or controlled substance addiction. In some embodiments of any of the methods described herein, the subject is entering into or participating in a medication monitoring program involving a controlled substance.

In some embodiments, the subject is a female (e.g., a pregnant female). In some embodiments, the subject is a male. For example, a subject in any of the methods described herein can be a child, an adolescent, a teenager, or an adult (a subject that greater than 18 years old, e.g., greater than 20 years old, greater than 25 years old, greater than 30 years old, greater than 35 years old, greater than 40 years old, greater than 45 years old, greater than 50 years old, greater than 55 years old, greater than 60 years old, greater than 65 years old, greater than 70 years old, greater than 75 years old, greater than 80 years old, greater than 90 years old, or greater than 100 years old). In some embodiments, the subject is a human.

In some embodiments, the subject is a genetic male (e.g., the subject has two distinct sex chromosomes (XY), the subject has an extra male chromosome (XYY), or the subject has two or more X chromosomes and a Y chromosome (e.g., XXY or XXXY)).

In any of the methods described herein, the subject may be employed by the military, may be a truck driver, a train engineer, a pilot, a medical professional (e.g., a physician, nurse, nurse's assistant, or a physician's assistant), or a pharmacist. In any of the methods described herein, the subject has a family history of illegal or controlled substance addiction. In any of the methods described herein, the subject can be identified as previously submitting a urine sample comprising, consisting essentially of, or consisting of an adulterated urine sample, a diluted urine sample, a urine sample originating from another subject, or a synthetic urine sample.

### Urine Samples

The methods described herein can include a step of providing a urine sample from a subject. In some embodiments, the methods described herein can further include a step of obtaining a urine sample from a subject. A urine sample is typically obtained using unwitnessed urine sample collection. As described herein, a urine sample can be a urine sample obtained from the subject.

In some embodiments, the urine sample originates from the subject. In other examples, the urine sample does not originate from the subject (e.g., a urine sample that includes (at least in part) a urine produced by another subject's body. For example, urine produced by another subject's body can be urine produced by a friend, a spouse, or a genetically-related family member (e.g., a parent or a sibling).

In some embodiments, a urine sample can include synthetic urine or a diluted urine sample (e.g., diluted with water). In some embodiments, a urine sample in the methods described herein can include an adulterated urine sample. For example, an adulterated urine sample can include genomic DNA from one or more of blood, hair, eyelashes, skin cells, saliva, semen, tears, mucus (e.g., eye or nose mucus), phlegm, or buccal cells (e.g., from the subject or from another subject).

A urine sample can have a volume of at least 1 mL (e.g., at least 2 mL, at least 3 mL, at least 4 mL, at least 5 mL, at least 6 mL, at least 7 mL, at least 8 mL, at least 9 mL, at least 10 mL, at least 12 mL, at least 14 mL, at least 16 mL, at least 18 mL, at least 20 mL, at least 22 mL, at least 24 mL, at least 26 mL, at least 28 mL, or at least 30 mL). In some embodiments, a urine sample can have a volume of between about 1 mL and about 30 mL, between about 5 mL and about 30 mL, between about 10 mL and about 30 mL, or between about 15 mL and about 30 mL. In some embodiments, a urine sample from a female subject can have a volume of at least 1 mL (e.g., at least 2 mL, at least 3 mL, at least 4 mL, at least 5 mL, at least 6 mL, at least 7 mL, at least 8 mL, at least 9 mL, at least 10 mL, or at least 15 mL). In some embodiments, a urine sample from a genetic male subject can have a volume of at least 10 mL, at least 15 mL, at least 20 mL, at least 25 mL, at least 30 mL, at least 35 mL, at least 40 mL, or at least 50 mL.

In some embodiments of any of the methods described herein, the urine sample can be stored, e.g., for at least 1 hour (e.g., at least 6 hours, at least 12 hours, at least 1 day, at least 2 days, at least 3 days, at least 4 days, at least 5 days, at least 6 days, or at least 7 days) at a temperature below 25° C. (e.g., at about 15° C., at about 10° C., at about 4° C., at about 0° C., at about −20° C., at about −40° C., at about −80° C., at about −86° C., or at about −196° C.) prior to enriching the urine sample for mammalian cells, if present.

In some embodiments, the urine sample can be centrifuged or clarified (e.g., by gravity) before any of the present methods are performed. In some embodiments, the urine sample can be filtered (e.g., to remove any bacterial cells, yeast cells, or soluble or insoluble protein aggregates) before any of the present methods are performed.

### Enrichment of a Urine Sample for Cells

The methods described herein include a step of enriching a urine sample (e.g., any of the urine samples described herein) for mammalian (e.g., human) cells (if present). A urine sample can be enriched for mammalian cells using a variety of different methods known in the art. In some embodiments, a urine sample can be centrifuged (e.g., ultracentrifuged) to pellet the mammalian cells present (if any) in the urine sample, the supernatant that is substantially free of mammalian cells aspirated or removed, and the resulting pellet optionally resuspended in a small volume of a buffer (e.g., a physiologically acceptable buffer or a cell lysis buffer, e.g., as the first step in the isolation of genomic DNA from the enriched sample). In some embodiments, a container holding a urine sample can be allowed to rest (without agitation), the supernatant that is substantially free of mammalian cells is aspirated from the container at a position that is opposite of the gravitational bottom of the container, and the resulting pellet containing mammalian cells (if present) is optionally resuspended in a small volume of buffer (e.g., a physiologically acceptable buffer or a cell lysis buffer, e.g., as the first step in the isolation of genomic DNA from the enriched sample).

In some embodiments, a urine sample can be enriched for mammalian cells by contacting the sample with a bead (e.g., a magnetic bead) coated with an antibody that specifically binds to mammalian cells (if present) in the urine sample. As is known in the art, the bound mammalian cells can be recovered from the bead in a small volume of buffer to yield an enriched sample that contains mammalian cells (if any) present in the urine sample. Similar beads that are covered with a fluorophore-labeled antibody that specifically binds to mammalian cells (if present) in the urine sample can be used to enrich any mammalian cells present in a urine sample through the use of fluorescence assisted cell sorting (FACS). Additional methods for enriching a urine sample for mammalian cells (if present) include the use of microfluidics. Such microfluidic methods are well known in the art (see, e.g., the methods described in Sethu et al., Anal. Chem. 78:5453-5461, 2006). One skilled in the art will appreciate that additional steps can be added to the methods described herein and other materials can be used to perform any of the steps of the methods described herein.

Isolating Genomic DNA from Enriched Samples

The methods provided herein further include a step of isolating any genomic DNA from the enriched sample to form an isolated genomic DNA test sample. A variety of methods for isolating genomic DNA from a sample (e.g., a sample containing mammalian cells enriched from a urine sample (if present)) are well known in the art. For example, a number of commercially available kits can be used to isolate genomic DNA from a sample containing mammalian cells (e.g., any of the enriched samples described herein). Non-limiting examples of commercially available kits for the isolation of genomic DNA from a sample containing mammalian cells include: Genomic DNA isolation kit (Abcam, Cambridge, U.K.), Genomic DNA Isolation Kit (Norgen Biotek Corp., Ontario, Canada), QIAmp DNA FFPE (Qiagen), QIAsymphony DSP DNA kits (Qiagen), REPLI-g Mini Kit (Qiagen), Generation Capture Plate Kit (Qiagen), Gentra Puregene Buccal Cell Kit (Qiagen), QI Amp 96 DNA Blood Kit (Qiagen), QIAmp DNA Mini kit (Qiagen), Biosprint 15 DNA Bloot Kit (Qiagen), Biosprint 96 DNA Blood Kit (Qiagen), MagAttract DNA Mini M48 Kit (Qiagen), QIAmp 96 DNA Swab BioRobot Kit, QIAmp DNA Blood BioRobot 9604 Kit (Qiagen), QIAmp DNA Investigator Kit (Qiagen), QIAmp DNA Micro Kit, ChargeSwitch® gDNA Normalized Buccal Cell Kits (Life Technologies), ChargeSwitch® gDNA Buccal Cell Kits (Life Technologies), Xtreme DNA Isolation Kit (Isohelix; Harrietsham, Kent, UK), DDK DNA Isolation Kit (Isohelix), XtraClean DNA kit (Isohelix), EzWay Buccal Swab DNA Isolation Kit (KOMABIOTECH, Seoul, Korea), QIAamp DNA Micro Kit (Qiagen), ZR Urine DNA Isolation Kit (Zymo Research), i-genomic Urine DNA Extraction Mini Kit (Intron Biotechnology, Korea), Abcam Urine Isolation Kit (Abcam, Cambridge, U.K.), and ReliaPrper Blood gDNA Miniprep System (Promega). Genomic DNA can be isolated from a sample (e.g., any of the enriched samples described herein) using these and other commercially available genomic DNA isolation kits by following the manufacturer's instructions.

An exemplary method for isolating genomic DNA from an enriched sample (e.g., any of the urine samples enriched for mammalian cells described herein) include the steps of: lysing the mammalian cells present (if any) in the enriched sample, precipitating proteins in the lysate, removing the supernatant, precipitating genomic DNA out of the supernatant, washing the genomic DNA pellet with ethanol, and rehydrating the genomic DNA pellet in a pharmaceutically acceptable buffer (e.g., sterile or filtered water, or a buffered solution). One skilled in the art will appreciate that additional steps can be added to the methods described herein and other materials can be used to perform any of the steps of the methods described herein.

### Amplification of Single Nucleotide Polymorphism (SNP Products)

The methods provided herein further include the steps of contacting a set of two or more oligonucleotide probes to the genomic DNA in the isolated genomic DNA test sample, wherein each of the two or more oligonucleotides probes hybridizes to a sequence of genomic DNA that is one nucleotide upstream of a different target single nucleotide polymorphism; and elongating the two or more oligonucleotide probes hybridized to the genomic DNA by one nucleotide using chain-terminating nucleotide(s) to generate a set of two or more SNP amplification products.

In some embodiments of these methods, the set of oligonucleotide probes includes, e.g., at least 2 oligonucleotide probes, at least 3 oligonucleotide probes, at least 4 oligonucleotide probes, at least 5 oligonucleotide probes, at least 6 oligonucleotide probes, at least 7 oligonucleotide probes, at least 8 oligonucleotide probes, at least 9 oligonucleotide probes, at least 10 oligonucleotide probes, at least 11 oligonucleotide probes, at least 12 oligonucleotide probes, at least 13 oligonucleotide probes, at least 14 oligonucleotide probes, at least 15 oligonucleotide probes, at least 16 oligonucleotide probes, at least 17 oligonucleotide probes, at least 18 oligonucleotide probes, at least 19 oligonucleotide probes, at least 20 oligonucleotide probes, at least 21 oligonucleotide probes, at least 22 oligonucleotide probes, at least 23 oligonucleotide probes, at least 24 oligonucleotide probes, at least 25 oligonucleotide probes, at least 26 oligonucleotide probes, at least 27 oligonucleotide probes, at least 28 oligonucleotide probes, at least 29 oligonucleotide probes, at least 30 oligonucleotide probes, at least 31 oligonucleotide probes, at least 32 oligonucleotide probes, at least 33 oligonucleotide probes, at least 34 oligonucleotide probes, at least 35 oligonucleotide probes, at least 36 oligonucleotide probes, at least 37 oligonucleotide probes, at least 38 oligonucleotide probes, at least 39 oligonucleotide probes, at least 40 oligonucleotide probes, at least 41 oligonucleotide probes, at least 42 oligonucleotide probes, at least 43 oligonucleotide probes, at least 44 oligonucleotide probes, at least 45 oligonucleotide probes, at least 46 oligonucleotide probes, at least 47 oligonucleotide probes, at least 48 oligonucleotide probes, at least 49 oligonucleotide probes, at least 50 oligonucleotide probes, at least 51 oligonucleotide probes, at least 52 oligonucleotide probes, at least 53 oligonucleotide probes, at least 54 oligonucleotide probes, at least 55 oligonucleotide probes, at least 56 oligonucleotide probes, at least 57 oligonucleotide probes, at least 58 oligonucleotide probes, at least 59 oligonucleotide probes, at least 60 oligonucleotide probes, at least 61 oligonucleotide probes, at least 62 oligonucleotides, at least 63 oligonucleotide probes, at least 64 oligonucleotide probes, at least 65 oligonucleotide probes, at least 66 oligonucleotide probes, at least 67 oligonucleotide probes, at least 68 oligonucleotide probes, at least 69 oligonucleotide probes, at least 70 oligonucleotide probes, at least 71 oligonucleotide probes, at least 72 oligonucleotide probes, at least 73 oligonucleotide probes, at least 74 oligonucleotide probes, at least 75 oligonucleotide probes, at least 76 oligonucleotide probes, at least 77 oligonucleotide probes, at least 78 oligonucleotide probes, at least 79 oligonucleotide probes, at least 80 oligonucleotide probes, at least 81 oligonucleotide probes, at least 82 oligonucleotide probes, at least 83 oligonucleotide probes, at least 84 oligonucleotide probes, at least 85 oligonucleotide probes, at least 86 oligonucleotide probes, at least 87 oligonucleotide probes, at least 88 oligonucleotide probes, at least 89 oligonucleotide probes, at least 90 oligonucleotide probes, at least 91 oligonucleotide probes, at least 92 oligonucleotide probes, at least 93 oligonucleotide probes, at least 94 oligonucleotide probes, at least 95 oligonucleotide probes, at least 96 oligonucleotide probes, at least 97 oligonucleotide probes, at least 98 oligonucleotide probes, at least 99 oligonucleotide probes, or at least 100 oligonucleotide probes.

In some embodiments of these methods, the set of oligonucleotide probes includes about 2 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, about 14 oligonucleotide probes, about 12 oligonucleotide probes, about 10 oligonucleotide probes, about 8 oligonucleotide probes, about 6 oligonucleotide probes, or about 4 oligonucleotide probes (inclusive); about 4 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, about 14 oligonucleotide probes, about 12 oligonucleotide probes, about 10 oligonucleotide probes, about 8 oligonucleotide probes, or about 6 oligonucleotide probes (inclusive); about 6 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, about 14 oligonucleotide probes, about 12 oligonucleotide probes, about 10 oligonucleotide probes, or about 8 oligonucleotide probes (inclusive); about 8 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, about 14 oligonucleotide probes, about 12 oligonucleotide probes, or about 10 oligonucleotide probes (inclusive); about 10 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, about 14 oligonucleotide probes, or about 12 oligonucleotide probes (inclusive); about 12 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, about 16 oligonucleotide probes, or about 14 oligonucleotide probes (inclusive); about 14 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, about 18 oligonucleotide probes, or about 16 oligonucleotide probes (inclusive); about 16 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, about 20 oligonucleotides probes, or about 18 oligonucleotide probes (inclusive); about 18 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, about 22 oligonucleotide probes, or about 20 oligonucleotides probes (inclusive); about 20 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, about 24 oligonucleotide probes, or about 22 oligonucleotide probes (inclusive); about 22 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, about 26 oligonucleotide probes, or about 24 oligonucleotide probes (inclusive); about 24 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, about 28 oligonucleotide probes, or about 26 oligonucleotide probes (inclusive); about 26 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, about 30 oligonucleotide probes, or about 28 oligonucleotide probes (inclusive); about 28 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, about 32 oligonucleotide probes, or about 30 oligonucleotide probes (inclusive); about 30 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, about 34 oligonucleotide probes, or about 32 oligonucleotide probes (inclusive); about 32 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, about 36 oligonucleotide probes, or about 34 oligonucleotide probes (inclusive); about 34 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, about 38 oligonucleotide probes, or about 36 oligonucleotide probes (inclusive); about 36 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, about 40 oligonucleotide probes, or about 38 oligonucleotide probes (inclusive); about 38 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, about 42 oligonucleotide probes, or about 40 oligonucleotide probes (inclusive); about 40 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, about 44 oligonucleotide probes, or about 42 oligonucleotide probes (inclusive); about 42 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, about 46 oligonucleotide probes, or about 44 oligonucleotide probes (inclusive); about 44 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, about 48 oligonucleotide probes, or about 46 oligonucleotide probes (inclusive); about 46 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, about 50 oligonucleotide probes, or about 48 oligonucleotide probes (inclusive); about 48 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, about 52 oligonucleotide probes, or about 50 oligonucleotide probes (inclusive); about 50 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, about 54 oligonucleotide probes, or about 52 oligonucleotide probes (inclusive); about 52 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, about 56 oligonucleotide probes, or about 54 oligonucleotide probes (inclusive); about 54 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, about 58 oligonucleotide probes, or about 56 oligonucleotide probes (inclusive); about 56 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, about 60 oligonucleotide probes, or about 58 oligonucleotide probes (inclusive); about 58 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, about 62 oligonucleotide probes, or about 60 oligonucleotide probes (inclusive); about 60 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, about 64 oligonucleotide probes, or about 62 oligonucleotide probes (inclusive); about 62 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, about 66 oligonucleotide probes, or about 64 oligonucleotide probes (inclusive); about 64 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, about 68 oligonucleotide probes, or about 66 oligonucleotide probes (inclusive); about 66 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, about 70 oligonucleotide probes, or about 68 oligonucleotide probes (inclusive); about 68 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, about 72 oligonucleotide probes, or about 70 oligonucleotide probes (inclusive); about 70 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, about 74 oligonucleotide probes, or about 72 oligonucleotide probes (inclusive); about 72 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, about 76 oligonucleotide probes, or about 74 oligonucleotide probes (inclusive); about 74 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, about 78 oligonucleotide probes, or about 76 oligonucleotide probes (inclusive); about 76 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, about 80 oligonucleotide probes, or about 78 oligonucleotide probes (inclusive); about 78 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, about 82 oligonucleotide probes, or about 80 oligonucleotide probes (inclusive); about 80 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, about 84 oligonucleotide probes, or about 82 oligonucleotide probes (inclusive); about 82 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, about 86 oligonucleotide probes, about 86 oligonucleotide probes, or about 84 oligonucleotide probes (inclusive); about 84 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, about 88 oligonucleotide probes, or about 86 oligonucleotide probes (inclusive); about 86 oligonucleotide probes to about 100 oligonucleotide probes, about 98 oligonucleotide probes, about 96 oligonucleotide probes, about 94 oligonucleotide probes, about 92 oligonucleotide probes, about 90 oligonucleotide probes, or about 88 oligonucleotide probes (inclusive); about 88 oligonucleotide probes to about 100 oligonucleotide probes, about 98 oligonucleotide probes, about 96 oligonucleotide probes, about 94 oligonucleotide probes, about 92 oligonucleotide probes, or about 90 oligonucleotide probes (inclusive); about 90 oligonucleotide probes to about 100 oligonucleotide probes, about 98 oligonucleotide probes, about 96 oligonucleotide probes, about 94 oligonucleotide probes, or about 92 oligonucleotide probes (inclusive); about 92 oligonucleotide probes to about 100 oligonucleotide probes, about 98 oligonucleotide probes, about 96 oligonucleotide probes, or about 94 oligonucleotide probes (inclusive); about 94 oligonucleotide probes to about 100 oligonucleotide probes, about 98 oligonucleotide probes, or about 96 oligonucleotide probes (inclusive); about 96 oligonucleotide probes to about 100 oligonucleotide probes or about 98 oligonucleotide probes (inclusive); or about 98 oligonucleotide probes to about 100 oligonucleotide probes (inclusive).

In some embodiments, each of the two or more oligonucleotide probes can have a length of 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, each of the two or more oligonucleotide probes can have a length of about 6 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, about 10 nucleotides, about 9 nucleotides, or about 8 nucleotides (inclusive); about 7 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, about 10 nucleotides, or about 9 nucleotides (inclusive); about 8 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, or about 10 nucleotides (inclusive); about 9 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, or about 11 nucleotides (inclusive); about 10 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, or about 12 nucleotides (inclusive); about 11 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, or about 13 nucleotides (inclusive); about 12 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, or about 14 nucleotides (inclusive); about 13 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, or about 15 nucleotides (inclusive); about 14 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, or about 16 nucleotides (inclusive); about 15 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, or about 17 nucleotides (inclusive); about 16 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, or about 18 nucleotides (inclusive); about 17 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, or about 19 nucleotides (inclusive); about 18 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, or about 20 nucleotides (inclusive); about 19 nucleotides to about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, or about 21 nucleotides (inclusive); about 20 nucleotides to about 30 nucleotides, about 29 nucleotides, about 28 nucleotides, about 27 nucleotides, about 26 nucleotides, about 25 nucleotides, about 24 nucleotides, about 23 nucleotides, or about 22 nucleotides (inclusive); about 21 nucleotides to about 30 nucleotides, about 29 nucleotides, about 28 nucleotides, about 27 nucleotides, about 26 nucleotides, about 25 nucleotides, about 24 nucleotides, or about 23 nucleotides (inclusive); about 22 nucleotides to about 30 nucleotides, about 29 nucleotides, about 28 nucleotides, about 27 nucleotides, about 26 nucleotides, about 25 nucleotides, or about 24 nucleotides (inclusive); about 23 nucleotides to about 30 nucleotides, about 29 nucleotides, about 28 nucleotides, about 27 nucleotides, about 26 nucleotides, or about 25 nucleotides (inclusive); about 24 nucleotides to about 30 nucleotides, about 29 nucleotides, about 28 nucleotides, about 27 nucleotides, or about 26 nucleotides (inclusive); about 25 nucleotides to about 30 nucleotides, about 29 nucleotides, about 28 nucleotides, or about 27 nucleotides (inclusive); about 26 nucleotides to about 30 nucleotides, about 29 nucleotides, or about 28 nucleotides (inclusive); about 27 nucleotides to about 30 nucleotides, or about 29 nucleotides (inclusive); or about 28 nucleotides to about 30 nucleotides (inclusive).

In some embodiments, each of the two or more oligonucleotide probes can include a detectable tag (e.g., positioned at its 5′ end). Non-limiting examples of detectable tags include a radioisotope (e.g., ³²P, ³⁵S, or ³H), a fluorophore (e.g., hydroxycoumarin, aminocoumarin, methoxycoumarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, fluorescein, BODIPY-FL, Cy2, Cy3, Cy3B, Cy3.5, Cy5, Cy5.5, Cy7, TRITC, X-Rhodamine, Lissamine Rhodamine B, Texas Red, Allophycocyanin (APC), and APC-Cy7 conjugates), or a fluorescent protein (e.g., mutant green fluorescent protein (e.g., Y66H, Y66W, Y66F, S65A, S65C, S65L, or S65T mutation), wildtype GFP, EBFP, EBFP2, azurite, GFPuv, T-Sapphire, Cerulean, mCFP, mTurquoise2, ECFP, CyPet, mKeima-Red, TagCFP, AmCyanl, mTFP1, Midoriishi Cyan, TurboGFP, TagGFP, Emerald, EGFP, Azami Green, ZsGreenl, TagYFP, EYFP, Topaz, Venus, mCitrine, YPet, TurboYFP, ZsYellowl, Kusabira Orange, mOrange, Allophycocyanin (APC), mKO, TurboRFP, tdTomato, TagRFP, DsRed monomer, DsRed2, mStrawberry, TurboFP602, AsRed2, mRFP1, J-Red, R-phycoerythrin, B-phycoerythrin, mCherry, HcRed1, Katusha, P3, Peridinin Chlorophyll, mKate, TurboFP635, mPlum, and mRaspberry.

In some embodiments, the set of SNP amplification products includes at least 2 SNP amplification products, at least 3 SNP amplification products, at least 4 SNP amplification products, at least 5 SNP amplification products, at least 6 SNP amplification products, at least 7 SNP amplification products, at least 8 SNP amplification products, at least 9 SNP amplification products, at least 10 SNP amplification products, at least 11 SNP amplification products, at least 12 SNP amplification products, at least 13 SNP amplification products, at least 14 SNP amplification products, at least 15 SNP amplification products, at least 16 SNP amplification products, at least 17 SNP amplification products, at least 18 SNP amplification products, at least 19 SNP amplification products, at least 20 SNP amplification products, at least 21 SNP amplification products, at least 22 SNP amplification products, at least 23 SNP amplification products, at least 24 SNP amplification products, at least 25 SNP amplification products, at least 26 SNP amplification products, at least 27 SNP amplification products, at least 28 SNP amplification products, at least 29 SNP amplification products, at least 30 SNP amplification products, at least 31 SNP amplification products, at least 32 SNP amplification products, at least 33 SNP amplification products, at least 34 SNP amplification products, at least 35 SNP amplification products, at least 36 SNP amplification products, at least 37 SNP amplification products, at least 38 SNP amplification products, at least 39 SNP amplification products, at least 40 SNP amplification products, at least 42 SNP amplification products, at least 44 SNP amplification products, at least 46 SNP amplification products, at least 48 SNP amplification products, at least 50 SNP amplification products, at least 52 SNP amplification products, at least 54 SNP amplification products, at least 56 SNP amplification products, at least 58 SNP amplification products, at least 60 SNP amplification products, at least 62 SNP amplification products, at least 64 SNP amplification products, at least 66 SNP amplification products, at least 68 SNP amplification products, at least 70 SNP amplification products, at least 72 SNP amplification products, at least 74 SNP amplification products, at least 76 SNP amplification products, at least 78 SNP amplification products, at least 80 SNP amplification products, at least 82 SNP amplification products, at least 84 SNP amplification products, at least 86 SNP amplification products, at least 88 SNP amplification products, at least 90 SNP amplification products, at least 92 SNP amplification products, at least 94 SNP amplification products, at least 96 SNP amplification products, at least 98 SNP amplification products, at least 100 SNP amplification products, at least 105 SNP amplification products, at least 110 SNP amplification products, at least 115 SNP amplification products, at least 120 SNP amplification products, at least 125 SNP amplification products, at least 130 SNP amplification products, at least 135 SNP amplification products, at least 140 SNP amplification products, at least 145 SNP amplification products, at least 150 SNP amplification products, at least 155 SNP amplification products, at least 160 SNP amplification products, at least 165 SNP amplification products, at least 170 SNP amplification products, at least 175 SNP amplification products, at least 180 SNP amplification products, at least 185 SNP amplification products, at least 190 SNP amplification products, at least 195 SNP amplification products, or at least 200 SNP amplification products.

In some embodiments, the set of SNP amplification products includes about 2 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, about 14 SNP amplification products, about 12 SNP amplification products, about 10 SNP amplification products, about 8 SNP amplification products, about 6 SNP amplification products, or about 4 SNP amplification products (inclusive); about 4 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, about 14 SNP amplification products, about 12 SNP amplification products, about 10 SNP amplification products, about 8 SNP amplification products, or about 6 SNP amplification products (inclusive); about 6 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, about 14 SNP amplification products, about 12 SNP amplification products, about 10 SNP amplification products, or about 8 SNP amplification products (inclusive); about 8 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, about 14 SNP amplification products, about 12 SNP amplification products, or about 10 SNP amplification products (inclusive); about 10 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, about 14 SNP amplification products, or about 12 SNP amplification products (inclusive); about 12 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, about 16 SNP amplification products, or about 14 SNP amplification products (inclusive); about 14 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, about 18 SNP amplification products, or about 16 SNP amplification products (inclusive); about 16 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, about 20 SNP amplification products, or about 18 SNP amplification products (inclusive); about 18 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, about 22 SNP amplification products, or about 20 SNP amplification products (inclusive); about 20 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, about 24 SNP amplification products, or about 22 SNP amplification products (inclusive); about 22 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, about 26 SNP amplification products, or about 24 SNP amplification products (inclusive); about 24 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, about 28 SNP amplification products, or about 26 SNP amplification products (inclusive); about 26 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, about 30 SNP amplification products, or about 28 SNP amplification products (inclusive); about 28 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, about 32 SNP amplification products, or about 30 SNP amplification products (inclusive); about 30 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, about 34 SNP amplification products, or about 32 SNP amplification products (inclusive); about 32 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, about 36 SNP amplification products, or about 34 SNP amplification products (inclusive); about 34 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, about 38 SNP amplification products, or about 36 SNP amplification products (inclusive); about 36 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, about 40 SNP amplification products, or about 38 SNP amplification products (inclusive); about 38 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, about 45 SNP amplification products, or about 40 SNP amplification products (inclusive); about 40 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products, or about 45 SNP amplification products (inclusive); about 45 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, about 55 SNP amplification products, about 50 SNP amplification products (inclusive); about 50 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, about 60 SNP amplification products, or about 55 SNP amplification products (inclusive); about 55 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, about 65 SNP amplification products, or about 60 SNP amplification products (inclusive); about 60 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, about 70 SNP amplification products, or about 65 SNP amplification products (inclusive); about 65 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, about 75 SNP amplification products, or about 70 SNP amplification products (inclusive); about 70 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, about 80 SNP amplification products, or about 75 SNP amplification products (inclusive); about 75 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, about 85 SNP amplification products, or about 80 SNP amplification products (inclusive); about 80 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, about 90 SNP amplification products, or about 85 SNP amplification products (inclusive); about 85 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, about 95 SNP amplification products, or about 90 SNP amplification products (inclusive); about 90 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, about 100 SNP amplification products, or about 95 SNP amplification products (inclusive); about 95 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, about 105 SNP amplification products, or about 100 SNP amplification products (inclusive); about 100 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, about 110 SNP amplification products, or about 105 SNP amplification products (inclusive); about 105 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, about 115 SNP amplification products, or about 110 SNP amplification products (inclusive); about 110 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, about 120 SNP amplification products, or about 115 SNP amplification products (inclusive); about 115 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, about 125 SNP amplification products, or about 120 SNP amplification products (inclusive); about 120 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, about 130 SNP amplification products, or about 125 SNP amplification products (inclusive); about 125 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, about 135 SNP amplification products, or about 130 SNP amplification products (inclusive); about 130 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, about 140 SNP amplification products, or about 135 SNP amplification products (inclusive); about 135 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, about 145 SNP amplification products, or about 140 SNP amplification products (inclusive); about 140 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, about 150 SNP amplification products, or about 145 SNP amplification products (inclusive); about 145 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products, or about 150 SNP amplification products (inclusive); about 150 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, about 160 SNP amplification products, about 155 SNP amplification products (inclusive); about 155 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, about 165 SNP amplification products, or about 160 SNP amplification products (inclusive); about 160 SNP amplification products to about 200 SNP amplification products, about 195 SNP amplification products, about 190 SNP amplification products, about 185 SNP amplification products, about 180 SNP amplification products, about 175 SNP amplification products, about 170 SNP amplification products, or about 165 SNP amplification products (inclusive); about 165 SNP amplification products to about 200 SNP amplification products, about 195 SNP amplification products, about 190 SNP amplification products, about 185 SNP amplification products, about 180 SNP amplification products, about 175 SNP amplification products, or about 170 SNP amplification products (inclusive); about 170 SNP amplification products to about 200 SNP amplification products, about 195 SNP amplification products, about 190 SNP amplification products, about 185 SNP amplification products, about 180 SNP amplification products, or about 175 SNP amplification products (inclusive); about 175 SNP amplification products to about 200 SNP amplification products, about 195 SNP amplification products, about 190 SNP amplification products, about 185 SNP amplification products, or about 180 SNP amplification products (inclusive); about 180 SNP amplification products to about 200 SNP amplification products, about 195 SNP amplification products, about 190 SNP amplification products, or about 185 SNP amplification products (inclusive); about 185 SNP amplification products to about 200 SNP amplification products, about 195 SNP amplification products, or about 190 SNP amplification products (inclusive); about 190 SNP amplification products to about 200 SNP amplification products, or about 195 SNP amplification products (inclusive); or about 195 SNP amplification products to about 200 SNP amplification products (inclusive).

In some examples, in the set of two or more SNP amplification products, each of the SNP amplification products can have a length of about 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, or 31 nucleotides. In some examples, in the set of two or more SNP amplification products, each of the SNP amplification products can have a length of about 7 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, about 10 nucleotides, or about 9 nucleotides (inclusive); about 8 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, or about 10 nucleotides (inclusive); about 9 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, or about 11 nucleotides (inclusive); about 10 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, about 13 nucleotides, or about 12 nucleotides (inclusive); about 11 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, about 14 nucleotides, or about 13 nucleotides (inclusive); about 12 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, about 15 nucleotides, or about 14 nucleotides (inclusive); about 13 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, about 16 nucleotides, or about 15 nucleotides (inclusive); about 14 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, about 17 nucleotides, or about 16 nucleotides (inclusive); about 15 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, about 18 nucleotides, or about 17 nucleotides (inclusive); about 16 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, about 19 nucleotides, or about 18 nucleotides (inclusive); about 17 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, about 20 nucleotides, or about 19 nucleotides (inclusive); about 18 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, about 21 nucleotides, or about 20 nucleotides (inclusive); about 19 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, about 22 nucleotides, or about 21 nucleotides (inclusive); about 20 nucleotides to about 31 nucleotides, about 30 nucleotides, about 29 nucleotides, about 28 nucleotides, about 27 nucleotides, about 26 nucleotides, about 25 nucleotides, about 24 nucleotides, about 23 nucleotides, or about 22 nucleotides (inclusive); about 21 nucleotides to about 31 nucleotides, about 30 nucleotides, about 29 nucleotides, about 28 nucleotides, about 27 nucleotides, about 26 nucleotides, about 25 nucleotides, about 24 nucleotides, or about 23 nucleotides (inclusive); about 22 nucleotides to about 31 nucleotides, about 30 nucleotides, about 29 nucleotides, about 28 nucleotides, about 27 nucleotides, about 26 nucleotides, about 25 nucleotides, or about 24 nucleotides (inclusive); about 23 nucleotides to about 31 nucleotides, about 30 nucleotides, about 29 nucleotides, about 28 nucleotides, about 27 nucleotides, about 26 nucleotides, or about 25 nucleotides (inclusive); about 24 nucleotides to about 31 nucleotides, about 30 nucleotides, about 29 nucleotides, about 28 nucleotides, about 27 nucleotides, about 26 nucleotides (inclusive); about 25 nucleotides to about 31 nucleotides, about 30 nucleotides, about 29 nucleotides, about 28 nucleotides, or about 27 nucleotides (inclusive); about 26 nucleotides to about 31 nucleotides, about 30 nucleotides, about 29 nucleotides, or about 28 nucleotides (inclusive); about 27 nucleotides to about 31 nucleotides, about 30 nucleotides, or about 29 nucleotides (inclusive); about 28 nucleotides to about 31 nucleotides, or about 30 nucleotides (inclusive); or about 29 nucleotides to about 31 nucleotides (inclusive).

In some embodiments of these methods, each of the two or more oligonucleotide probes hybridizes (e.g., specifically hybridizes) to a sequence of genomic DNA that is one nucleotide upstream of a target SNP having a minor allele frequency of >0.4 (e.g., a minor allele frequency of >0.45, a minor allele frequency of >0.50, a minor allele frequency of >0.55, or a minor allele frequency of >0.60).

In some embodiments, at least one (e.g., at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, two, three, four, five, six, seven, eight, nine, ten, eleven, twelve, thirteen, fourteen, fifteen, sixteen, seventeen, eighteen, or nineteen) target SNP can be selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments, at least one (e.g., at least two, at least three, at least four, at least five, at least six, at least seven, at least eight, at least nine, at least ten, at least eleven, at least twelve, at least thirteen, two, three, four, five, six, seven, eight, nine, ten, eleven, twelve, thirteen, or fourteen) target SNP can be selected from the group of: rs7520386, rs560681, rs9951171, rs1058083, rs1358856, rs214955, rs740598, rs279844, rs13218440, rs2272998, rs12997453, rs13134862, rs13182883, and rs1410059.

In some embodiments, where the subject a genetic male, at least one (e.g., at least two, at least three, or at least four) target SNP is located on a Y chromosome.

In some embodiments, where the set of oligonucleotide probes includes at least three (e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least three different chromosomes.

In some embodiments, where the set of oligonucleotide probes includes at least four (e.g., at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least four different chromosomes.

In some embodiments, where the set of oligonucleotide probes includes at least five (e.g., at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least five different chromosomes.

In some embodiments, where the set of oligonucleotide probes includes at least six (e.g., at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least six different chromosomes.

In some embodiments, where the set of oligonucleotide probes includes at least seven (e.g., at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least seven different chromosomes.

In some embodiments, where the set of oligonucleotide probes includes at least eight (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least eight different chromosomes.

In some embodiments, where the set of oligonucleotide probes includes at least nine (e.g., at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least nine different chromosomes.

In some embodiments, where the set of oligonucleotide probes includes at least ten (e.g., at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least ten different chromosomes.

In some embodiments, where the set of oligonucleotide probes includes at least eleven (e.g., at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least eleven different chromosomes.

In some embodiments, where the set of oligonucleotide probes includes at least twelve (e.g., at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least twelve different chromosomes.

In some embodiments, where the set of oligonucleotide probes includes at least thirteen (e.g., at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least thirteen different chromosomes.

In some embodiments, where the set of oligonucleotide probes includes at least fourteen (e.g., at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least fourteen different chromosomes.

In some embodiments, where the set of oligonucleotide probes includes at least fifteen (e.g., at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least fifteen different chromosomes.

In some embodiments, where the set of oligonucleotide probes includes at least sixteen (e.g., at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least sixteen different chromosomes.

In some embodiments, where the set of oligonucleotide probes includes at least seventeen (e.g., at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least seventeen different chromosomes.

In some embodiments, where the set of oligonucleotide probes includes at least eighteen (e.g., at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least eighteen different chromosomes.

In some embodiments, where the set of oligonucleotide probes includes at least nineteen (e.g., at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least nineteen different chromosomes.

In some embodiments, where the set of oligonucleotide probes includes at least twenty (e.g., at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 94, at least 96, at lease 98, or at least 100) oligonucleotide probes, the target SNPs can include at least one target SNP from at least twenty different chromosomes.

In some embodiments, the elongating step can include: (l) denaturing the genomic DNA at about 85° C. to about 96° C. (inclusive) (e.g., about 85° C. to about 95° C., about 94° C., about 93° C., about 92° C., about 91° C., about 90° C., about 89° C., about 88° C., or about 87° C. (inclusive); about 86° C. to about 96° C., about 95° C., about 94° C., about 93° C., about 92° C., about 91° C., about 90° C., about 89° C., or about 88° C. (inclusive); about 87° C. to about 96° C., about 95° C., about 94° C., about 93° C., about 92° C., about 91° C., about 90° C., or about 89° C. (inclusive); about 88° C. to about 96° C., about 95° C., about 94° C., about 93° C., about 92° C., about 91° C., or about 90° C. (inclusive); about 89° C. to about 96° C., about 95° C., about 94° C., about 93° C., about 92° C., or about 91° C. (inclusive); about 90° C. to about 96° C., about 95° C., about 94° C., about 93° C., or about 92° C. (inclusive); about 91° C. to about 96° C., about 95° C., about 94° C., or about 93° C. (inclusive); about 92° C. to about 96° C., about 95° C., or about 94° C. (inclusive); or about 93° C. to about 96° C. or about 95° C. (inclusive); or about 94° C. to about 96° C. (inclusive)) for about 2 seconds to about 60 seconds (e.g., about 2 seconds to about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, about 8 seconds, about 6 seconds, or about 4 seconds (inclusive); about 4 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, about 8 seconds, or about 6 seconds (inclusive); about 6 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, or about 8 seconds (inclusive); about 8 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, or about 10 seconds (inclusive); about 10 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, or about 12 seconds (inclusive); about 12 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, or about 14 seconds (inclusive); about 14 seconds to about 60 seconds, about 55 seconds, about 50 seconds, about 45 seconds, about 40 seconds, about 35 seconds, about 30 seconds, about 25 seconds, about 20 seconds, about 18 seconds, or about 16 seconds (inclusive); about 16 seconds to about 60 seconds, about 55 seconds, about 50 seconds, about 45 seconds, about 40 seconds, about 35 seconds, about 30 seconds, about 25 seconds, about 20 seconds, or about 18 seconds (inclusive); about 18 seconds to about 60 seconds, about 55 seconds, about 50 seconds, about 45 seconds, about 40 seconds, about 35 seconds, about 30 seconds, about 25 seconds, or about 20 seconds (inclusive); about 20 seconds to about 60 seconds, about 55 seconds, about 50 seconds, about 45 seconds, about 40 seconds, about 35 seconds, about 30 seconds, or about 25 seconds (inclusive); about 25 seconds to about 60 seconds, about 55 seconds, about 50 seconds, about 45 seconds, about 40 seconds, about 35 seconds, or about 30 seconds (inclusive); about 30 seconds to about 60 seconds, about 55 seconds, about 50 seconds, about 45 seconds, about 40 seconds, or about 35 seconds (inclusive); about 35 seconds to about 60 seconds, about 55 seconds, about 50 seconds, about 45 seconds, or about 40 seconds (inclusive); about 40 seconds to about 60 seconds, about 55 seconds, about 50 seconds, or about 45 seconds (inclusive); about 45 seconds to about 60 seconds, about 55 seconds, or about 50 seconds (inclusive); about 50 seconds to about 60 seconds or about 55 seconds (inclusive); or about 55 seconds to about 60 seconds (inclusive)); (2) annealing the two or more oligonucleotide probes at a temperature of about 46° C. to about 60° C. (e.g., about 46° C. to about 59° C., about 58° C., about 57° C., about 56° C., about 55° C., about 54° C., about 53° C., about 52° C., about 51° C., about 50° C., about 49° C., or about 48° C. (inclusive); about 47° C. to about 60° C., about 59° C., about 58° C., about 57° C., about 56° C., about 55° C., about 54° C., about 53° C., about 52° C., about 51° C., about 50° C., or about 49° C. (inclusive); about 48° C. to about 60° C., about 59° C., about 58° C., about 57° C., about 56° C., about 55° C., about 54° C., about 53° C., about 52° C., about 51° C., or about 50° C. (inclusive); about 49° C. to about 60° C., about 59° C., about 58° C., about 57° C., about 56° C., about 55° C., about 54° C., about 53° C., about 52° C., or about 51° C. (inclusive); about 50° C. to about 60° C., about 59° C., about 58° C., about 57° C., about 56° C., about 55° C., about 54° C., about 53° C., or about 52° C. (inclusive); about 51° C. to about 60° C., about 59° C., about 58° C., about 57° C., about 56° C., about 55° C., about 54° C., or about 53° C. (inclusive); about 52° C. to about 60° C., about 59° C., about 58° C., about 57° C., about 56° C., about 55° C., or about 54° C. (inclusive); about 53° C. to about 60° C., about 59° C., about 58° C., about 57° C., about 56° C., or about 55° C. (inclusive); about 54° C. to about 60° C., about 59° C., about 58° C., about 57° C., or about 56° C. (inclusive); about 55° C. to about 60° C., about 59° C., about 58° C., or about 57° C. (inclusive); about 56° C. to about 60° C., about 59° C., or about 58° C. (inclusive); about 57° C. to about 60° C. or about 59° C. (inclusive); or about 58° C. to about 60° C. (inclusive)) for about 2 seconds to about 60 seconds (e.g., about 2 seconds to about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, about 8 seconds, about 6 seconds, or about 4 seconds (inclusive); about 4 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, about 8 seconds, or about 6 seconds (inclusive); about 6 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, about 12 seconds, about 10 seconds, or about 8 seconds (inclusive); about 10 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, about 14 seconds, or about 12 seconds (inclusive); about 12 seconds to about 60 seconds, about 55 seconds, about 50 seconds, about 45 seconds, about 40 seconds, about 35 seconds, about 30 seconds, about 25 seconds, about 20 seconds, about 18 seconds, about 16 seconds, or about 14 seconds (inclusive); about 14 seconds to about 60 seconds, about 55 seconds, about 50 seconds, about 45 seconds, about 40 seconds, about 35 seconds, about 30 seconds, about 25 seconds, about 20 seconds, about 18 seconds, or about 16 seconds (inclusive); about 16 seconds to about 60 seconds, about 55 seconds, about 50 seconds, about 45 seconds, about 40 seconds, about 35 seconds, about 30 seconds, about 25 seconds, about 20 seconds, or about 18 seconds (inclusive); about 18 seconds to about 60 seconds, about 55 seconds, about 50 seconds, about 45 seconds, about 40 seconds, about 35 seconds, about 30 seconds, about 25 seconds, or about 20 seconds (inclusive); about 20 seconds to about 60 seconds, about 55 seconds, about 50 seconds, about 45 seconds, about 40 seconds, about 35 seconds, about 30 seconds, or about 25 seconds (inclusive); about 25 seconds to about 60 seconds, about 55 seconds, about 50 seconds, about 45 seconds, about 40 seconds, about 35 seconds, or about 30 seconds (inclusive); about 30 seconds to about 60 seconds, about 55 seconds, about 50 seconds, about 45 seconds, about 40 seconds, or about 35 seconds (inclusive); about 35 seconds to about 60 seconds, about 55 seconds, about 50 seconds, about 45 seconds, or about 40 seconds (inclusive); about 40 seconds to about 60 seconds, about 55 seconds, about 50 seconds, or about 45 seconds (inclusive); about 45 seconds to about 60 seconds, about 55 seconds, or about 50 seconds (inclusive); about 50 seconds to about 60 seconds or about 55 seconds (inclusive); or about 55 second to about 60 seconds (inclusive)); and (3) performing extension at about 70° C. to about 82° C. (e.g., about 70° C. to about 81° C., about 80° C., about 79° C., about 78° C., about 77° C., about 76° C., about 75° C., about 74° C., about 73° C., or about 72° C. (inclusive); about 71° C. to about 82° C., about 81° C., about 80° C., about 79° C., about 78° C., about 77° C., about 76° C., about 75° C., about 74° C., or about 73° C. (inclusive); about 72° C. to about 82° C., about 81° C., about 80° C., about 79° C., about 78° C., about 77° C., about 76° C., about 75° C., or about 74° C. (inclusive); about 73° C. to about 82° C., about 81° C., about 80° C., about 79° C., about 78° C., about 77° C., about 76° C., or about 75° C. (inclusive); about 74° C. to about 82° C., about 81° C., about 80° C., about 79° C., about 78° C., about 77° C., or about 76° C. (inclusive); about 75° C. to about 82° C., about 81° C., about 80° C., about 79° C., about 78° C., or about 77° C. (inclusive); about 76° C. to about 82° C., about 81° C., about 80° C., about 79° C., or about 78° C. (inclusive); about 77° C. to about 82° C., about 81° C., about 80° C., or about 79° C. (inclusive); about 78° C. to about 82° C., about 81° C., or about 80° C. (inclusive); about 79° C. to about 82° C. or about 81° C. (inclusive); or about 80° C. to about 82° C. (inclusive)) for about 5 seconds to about 60 seconds (e.g., about 5 seconds to about 55 seconds, about 50 seconds, about 45 seconds, about 40 seconds, about 35 seconds, about 30 seconds, about 25 seconds, about 20 seconds, about 15 seconds, or about 10 seconds (inclusive); about 10 seconds to about 60 seconds, about 55 seconds, about 50 seconds, about 45 seconds, about 40 seconds, about 35 seconds, about 30 seconds, about 25 seconds, about 20 seconds, or about 15 seconds (inclusive); about 15 seconds to about 60 seconds, about 55 seconds, about 50 seconds, about 45 seconds, about 40 seconds, about 35 seconds, about 30 seconds, about 25 seconds, or about 20 seconds (inclusive); about 20 seconds to about 60 seconds, about 55 seconds, about 50 seconds, about 45 seconds, about 40 seconds, about 35 seconds, about 30 seconds, or about 25 seconds (inclusive); about 25 seconds to about 60 seconds, about 55 seconds, about 50 seconds, about 45 seconds, about 40 seconds, about 35 seconds, or about 30 seconds (inclusive); about 35 seconds to about 60 seconds, about 55 seconds, about 50 seconds, about 45 seconds, or about 40 seconds (inclusive); about 40 seconds to about 60 seconds, about 55 seconds, about 50 seconds, or about 45 seconds (inclusive); about 45 seconds to about 60 seconds, about 55 seconds, or about 50 seconds (inclusive); about 50 seconds to about 60 seconds or about 55 seconds (inclusive); or about 55 seconds to about 60 seconds (inclusive)). In some embodiments, the sequential steps (l), (2) and (3) can be repeated for about 2 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, about 14 cycles, about 12 cycles, about 10 cycles, about 8 cycles, about 6 cycles, or about 4 cycles (inclusive); about 4 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, about 14 cycles, about 12 cycles, about 10 cycles, about 8 cycles, or about 6 cycles (inclusive); about 6 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, about 14 cycles, about 12 cycles, about 10 cycles, or about 8 cycles (inclusive); about 8 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, about 14 cycles, about 12 cycles, or about 10 cycles (inclusive); about 10 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, about 14 cycles, or about 12 cycles (inclusive); about 12 cycles to about 60 cycles, about 55 cycles, about 50 cycles, about 45 cycles, about 40 cycles, about 35 cycles, about 30 cycles, about 25 cycles, about 20 cycles, about 18 cycles, about 16 cycles, or about 14 cycles (inclusive); about 14 cycles to about 60 cycles, about 55 cycles, about 50 cycles, about 45 cycles, about 40 cycles, about 35 cycles, about 30 cycles, about 25 cycles, about 20 cycles, about 18 cycles, or about 16 cycles (inclusive); about 16 cycles to about 60 cycles, about 55 cycles, about 50 cycles, about 45 cycles, about 40 cycles, about 35 cycles, about 30 cycles, about 25 cycles, about 20 cycles, or about 18 cycles (inclusive); about 18 cycles to about 60 cycles, about 55 cycles, about 50 cycles, about 45 cycles, about 40 cycles, about 35 cycles, about 30 cycles, about 25 cycles, about 20 cycles (inclusive); about 20 cycles to about 60 cycles, about 55 cycles, about 50 cycles, about 45 cycles, about 40 cycles, about 35 cycles, about 30 cycles, or about 25 cycles (inclusive); about 25 cycles to about 60 cycles, about 55 cycles, about 50 cycles, about 45 cycles, about 40 cycles, about 35 cycles, or about 30 cycles (inclusive); about 30 cycles to about 60 cycles, about 55 cycles, about 50 cycles, about 45 cycles, about 40 cycles, or about 35 cycles (inclusive); about 35 cycles to about 60 cycles, about 55 cycles, about 50 cycles, about 45 cycles, or about 40 cycles (inclusive); about 40 cycles to about 60 cycles, about 55 cycles, about 50 cycles, or about 45 cycles (inclusive); about 45 cycles to about 60 cycles, about 55 cycles, or about 50 cycles (inclusive); about 50 cycles to about 60 cycles or about 55 cycles (inclusive); or about 55 cycles to about 60 cycles (inclusive).

In some embodiments, the elongating can further include one or more of: an initial denaturation step prior to the denaturing step (l), performed at about 85° C. to about 96° C. (e.g., about 85° C. to about 95° C., about 94° C., about 93° C., about 92° C., about 91° C., about 90° C., about 89° C., about 88° C., or about 87° C. (inclusive); about 86° C. to about 96° C., about 95° C., about 94° C., about 93° C., about 92° C., about 91° C., about 90° C., about 89° C., or about 88° C. (inclusive); about 87° C. to about 96° C., about 95° C., about 94° C., about 93° C., about 92° C., about 91° C., about 90° C., or about 89° C. (inclusive); about 88° C. to about 96° C., about 95° C., about 94° C., about 93° C., about 92° C., about 91° C., or about 90° C. (inclusive); about 89° C. to about 96° C., about 95° C., about 94° C., about 93° C., about 92° C., or about 91° C. (inclusive); about 90° C. to about 96° C., about 95° C., about 94° C., about 93° C., or about 92° C. (inclusive); about 91° C. to about 96° C., about 95° C., about 94° C., or about 93° C. (inclusive); about 92° C. to about 96° C., about 95° C., or about 94° C. (inclusive); about 93° C. to about 96° C. or about 95° C. (inclusive); or about 94° C. to about 96° C. (inclusive)) for about 15 seconds to about 60 seconds (e.g., about 15 seconds to about 55 seconds, about 50 seconds, about 45 seconds, about 40 seconds, about 35 seconds, about 30 seconds, about 25 seconds, or about 20 seconds (inclusive); about 20 seconds to about 60 seconds, about 55 seconds, about 50 seconds, about 45 seconds, about 40 seconds, about 35 seconds, about 30 seconds, or about 25 seconds (inclusive); about 25 seconds to about 60 seconds, about 55 seconds, about 50 seconds, about 45 seconds, about 40 seconds, about 35 seconds, or about 30 seconds (inclusive); about 30 seconds to about 60 seconds, about 55 seconds, about 50 seconds, about 45 seconds, about 40 seconds, or about 35 seconds (inclusive); about 35 seconds to about 60 seconds, about 55 seconds, about 50 seconds, about 45 seconds, or about 40 seconds (inclusive); about 40 seconds to about 60 seconds, about 55 seconds, about 50 seconds, or about 45 seconds (inclusive); about 45 seconds to about 60 seconds, about 55 seconds, or about 50 seconds (inclusive); about 50 seconds to about 60 seconds or about 55 seconds (inclusive)); a final extension step performed at about 70° C. to about 82° C. (e.g., about 70° C. to about 81° C., about 80° C., about 79° C., about 78° C., about 77° C., about 76° C., about 75° C., about 74° C., about 73° C., or about 72° C. (inclusive); about 71° C. to about 82° C., about 81° C., about 80° C., about 79° C., about 78° C., about 77° C., about 76° C., about 75° C., about 74° C., or about 73° C. (inclusive); about 72° C. to about 82° C., about 81° C., about 80° C., about 79° C., about 78° C., about 77° C., about 76° C., about 75° C., or about 74° C. (inclusive); about 73° C. to about 82° C., about 81° C., about 80° C., about 79° C., about 78° C., about 77° C., about 76° C., or about 75° C. (inclusive); about 74° C. to about 82° C., about 81° C., about 80° C., about 79° C., about 78° C., about 77° C., or about 76° C. (inclusive); about 75° C. to about 82° C., about 81° C., about 80° C., about 79° C., about 78° C., or about 77° C. (inclusive); about 76° C. to about 82° C., about 81° C., about 80° C., about 79° C., or about 78° C. (inclusive); about 77° C. to about 82° C., about 81° C., about 80° C., or about 79° C. (inclusive); about 78° C. to about 82° C., about 81° C., or about 80° C. (inclusive); about 79° C. to about 82° C. or about 81° C. (inclusive); or about 80° C. to about 82° C. (inclusive)) for about 1 minute to about 5 minutes (e.g., about 1 minute to about 4 minutes, about 3 minutes, or about 2 minutes (inclusive); about 2 minutes to about 5 minutes, about 4 minutes, or about 3 minutes (inclusive); about 3 minutes to about 5 minutes or about 4 minutes (inclusive); or about 4 minutes to about 5 minutes (inclusive)); and/or a hold step at 4° C. for about or at least 2 minutes (e.g., about or at least 4 minutes, about or at least 6 minutes, about or at least 8 minutes, about or at least 10 minutes, about or at least 20 minutes, about or at least 30 minutes, about or at least 40 minutes, about or at least 50 minutes, about or at least 1 hour, about or at least 2 hours, about or at least 3 hours, about or at least 4 hours, about or at least 5 hours, about or at least 6 hours, about or at least 8 hours, about or at least 10 hours, about or at least 12 hours, about or at least 18 hours, about or at least 24 hours, about or at least 36 hours, about or at least 48 hours, about or at least 60 hours, about or at least 72 hours, about or at least 84 hours, or about or at least 96 hours). One skilled in the art will appreciate that additional steps can be added to the elongation step described herein and other materials can be used to perform the elongation step.

The assay used to elongate the two or more oligonucleotide probes (e.g., at least two oligonucleotide probes, at least three oligonucleotide probes, at least four oligonucleotide probes, at least five oligonucleotide probes, at least six oligonucleotide probes, at least seven oligonucleotide probes, at least eight oligonucleotide probes, at least nine oligonucleotide probes, at least ten oligonucleotide probes, at least eleven oligonucleotide probes, at least twelve oligonucleotide probes, at least thirteen oligonucleotide probes, at least fourteen oligonucleotide probes, at least fifteen oligonucleotide probes, at least sixteen oligonucleotide probes, at least seventeen oligonucleotide probes, at least eighteen oligonucleotide probes, at least 19 oligonucleotide probes, at least 20 oligonucleotide probes, at least 22 oligonucleotide probes, at least 24 oligonucleotide probes, at least 26 oligonucleotide probes, about least 28 oligonucleotide probes, at least 30 oligonucleotide probes, at least 32 oligonucleotide probes, at least 34 oligonucleotide probes, at least 36 oligonucleotide probes, at least 38 oligonucleotide probes, at least 40 oligonucleotide probes, at least 42 oligonucleotide probes, at least 44 oligonucleotide probes, at least 46 oligonucleotide probes, at least 48 oligonucleotide probes, at least 50 oligonucleotide probes, at least 52 oligonucleotide probes, at least 54 oligonucleotides probes, at least 56 oligonucleotide probes, at least 58 oligonucleotide probes, at least 60 oligonucleotide probes, at least 62 oligonucleotide probes, at least 64 oligonucleotide probes, at least 66 oligonucleotide probes, at least 68 oligonucleotide probes, at least 70 oligonucleotide probes, at least 72 oligonucleotide probes, at least 74 oligonucleotide probes, at least 76 oligonucleotide probes, at least 78 oligonucleotide probes, at least 80 oligonucleotide probes, at least 82 oligonucleotide probes, at least 84 oligonucleotide probes, at least 86 oligonucleotide probes, at least 88 oligonucleotide probes, at least 90 oligonucleotide probes, at least 92 oligonucleotide probes, at least 94 oligonucleotide probes, at least 96 oligonucleotide probes, at least 98 oligonucleotide probes, or at least 100 oligonucleotide) to generate a set of two or more SNP amplification products can be, e.g., a polymerase chain reaction (PCR) assay (e.g., a locus-specific primer extension reaction (iPLEX assay), a real-time PCR assay or any of the other assays for genotyping a SNP described herein. See e.g., Fakruddin, et al., J. Pharm. Bioallied Sci. 5(4): 245-252; and Gabriel et al., Current Protocols in Human Genetics, Chapter 2: Unit 2.12, 2009.

In some embodiments, the chain-terminating nucleotide(s) is/are 2′,3′-dideoxynucleotide(s) (ddNTP(s)), e.g., ddATP, ddCTP, ddGTP, and ddTTP. A ddNTP is a sugar modified nucleoside triphosphate, where the 2′ and 3′ hydroxyl groups are absent. In the absence of the 2′ and 3′ hydroxyl groups, polymerases are unable to extend, which causes a chain termination.

In some embodiments, the chain-terminating nucleotide(s) is/are 3′-azido-nucleotide derivative(s) (3′-azido-ddNTP(s)), e.g., 3′-azido-ddATP, 3′-azido-ddTTP, 3′-azido-ddCTP, and 3′-azido-ddGTP.

In some embodiments, the chain-terminating nucleotide(s) is/are 3′amino-nucleotide derivative(s) (3′-amino-ddNTP(s)), e.g., 3′-amino-ddATP, 3′-amino-ddTTP, 3′-amino-ddCTP, and 3′-amino-ddGTP.

In some embodiments, the chain-terminating nucleotide(s) is/are mass-modified dideoxynucleotide terminators (ddNTP(s)). In some embodiments, the mass-modified ddNTPs are biotinylated. In some embodiments, the biotinylated mass-modified ddNTPs are selected from the group of: biotin-11-ddATP, biotin-11-ddCTP, biotin-11-ddGTP, biotin-11-ddUTP, or biotin-16-ddUTP (see, e.g., Edwards et al., Nucleic Acid Research 29(21): e104, 2001). In some embodiments, where the mass-modified ddNTPs are biotinylated, the set of SNP amplification products is readily separated from non-elongated oligonucleotide probes that are not biotinylated, following incubation with streptavidin-coated magnetic particles.

Additional examples of chain-terminating nucleotides are described in, e.g., U.S. Pat. No. 5,547,859. Additional examples of chain-terminating nucleotides are known in the art.

### Pre-Amplification of Genomic DNA

Some embodiments of any of the methods described herein can further include, between steps (c) and (d), a step of performing a pre-amplification step. In some embodiments, the pre-amplification step includes hybridization of two or more (e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more 37 or more, 38 or more, 39 or more, 40 or more, 42 or more, 44 or more, 46 or more, 48 or more, 50 or more, 52 or more, 54 or more, 56 or more, 58 or more, 60 or more, 62 or more, 64 or more, 66 or more, 68 or more, 70 or more, 72 or more, 74 or more, 76 or more, 78 or more, 80 or more, 82 or more, 84 or more, 86 or more, 88 or more, 90 or more, 92 or more, 94 or more, 96 or more, 98 or more, or 100 or more) pairs of pre-amplification forward and reverse primers, where each pair of pre-amplification forward and reverse primers is designed to amplify about 100 to about 500 nucleotides (e.g., about 100 nucleotides to about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, about 220 nucleotides, about 200 nucleotides, about 180 nucleotides, about 160 nucleotides, about 140 nucleotides, or about 120 nucleotides (inclusive); about 120 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, about 220 nucleotides, about 200 nucleotides, about 180 nucleotides, about 160 nucleotides, or about 140 nucleotides (inclusive); about 140 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, about 220 nucleotides, about 200 nucleotides, about 180 nucleotides, or about 160 nucleotides (inclusive); about 160 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, about 220 nucleotides, about 200 nucleotides, or about 180 nucleotides (inclusive); about 180 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, about 220 nucleotides, or about 200 nucleotides (inclusive); about 200 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, about 240 nucleotides, or about 220 nucleotides (inclusive); about 220 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, about 260 nucleotides, or about 240 nucleotides (inclusive); about 240 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, about 280 nucleotides, or about 260 nucleotides (inclusive); about 260 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, about 300 nucleotides, or about 280 nucleotides (inclusive); about 280 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, about 320 nucleotides, or about 300 nucleotides (inclusive); about 300 nucleotides to about 500 nucleotides, about 480 nucleotides, about 460 nucleotides, about 440 nucleotides, about 420 nucleotides, about 400 nucleotides, about 380 nucleotides, about 360 nucleotides, about 340 nucleotides, or about 320 nucleotides (inclusive); about 320 nucleotides to about 500 nucleotides, about 480 nucleotides, about 460 nucleotides, about 440 nucleotides, about 420 nucleotides, about 400 nucleotides, about 380 nucleotides, about 360 nucleotides, or about 340 nucleotides (inclusive); about 340 nucleotides to about 500 nucleotides, about 480 nucleotides, about 460 nucleotides, about 440 nucleotides, about 420 nucleotides, about 400 nucleotides, about 380 nucleotides, or about 360 nucleotides (inclusive); about 360 nucleotides to about 500 nucleotides, about 480 nucleotides, about 460 nucleotides, about 440 nucleotides, about 420 nucleotides, about 400 nucleotides, or about 380 nucleotides (inclusive); about 380 nucleotides to about 500 nucleotides, about 480 nucleotides, about 460 nucleotides, about 440 nucleotides, about 420 nucleotides, or about 400 nucleotides (inclusive); about 400 nucleotides to about 500 nucleotides, about 480 nucleotides, about 460 nucleotides, about 440 nucleotides, or about 420 nucleotides (inclusive); about 420 nucleotides to about 500 nucleotides, about 480 nucleotides, about 460 nucleotides, or about 440 nucleotides (inclusive); about 440 nucleotides to about 500 nucleotides, about 480 nucleotides, or about 460 nucleotides (inclusive); about 460 nucleotides to about 500 nucleotides or about 480 nucleotides (inclusive); or about 480 nucleotides to about 500 nucleotides (inclusive)) of genomic DNA that contains one of the different target SNPs, where the pre-amplification forward and reverse primer in each of the pairs of pre-amplification primers contains (i) a sequence of about 10 to about 30 contiguous nucleotides (e.g., about 10 contiguous nucleotides to about 28 contiguous nucleotides, about 26 nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, or about 12 contiguous nucleotides (inclusive); about 12 contiguous nucleotides to about 30 contiguous nucleotides, about 28 contiguous nucleotides, about 26 nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 30 contiguous nucleotides, about 28 contiguous nucleotides, about 26 nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 30 contiguous nucleotides, about 28 contiguous nucleotides, about 26 nucleotides, about 24 contiguous nucleotides, about 22 contiguous nucleotides, about 20 contiguous nucleotides, or about 18 contiguous nucleotides (inclusive); about 18 contiguous nucleotides to about 30 contiguous nucleotides, about 28 contiguous nucleotides, about 26 nucleotides, about 24 contiguous nucleotides, about 22 contiguous nucleotides, or about 20 contiguous nucleotides (inclusive); about 20 contiguous nucleotides to about 30 contiguous nucleotides, about 28 contiguous nucleotides, about 26 nucleotides, about 24 contiguous nucleotides, or about 22 contiguous nucleotides (inclusive); about 22 contiguous nucleotides to about 30 contiguous nucleotides, about 28 contiguous nucleotides, about 26 nucleotides, or about 24 contiguous nucleotides (inclusive); about 24 contiguous nucleotides to about 30 contiguous nucleotides, about 28 contiguous nucleotides, or about 26 contiguous nucleotides (inclusive); about 26 contiguous nucleotides to about 30 contiguous nucleotides or about 28 contiguous nucleotides (inclusive); or about 28 contiguous nucleotides to about 30 contiguous nucleotides (inclusive)) that is complementary to a sequence in the genomic DNA and (ii) a tag sequence of about 5 contiguous nucleotides to about 25 contiguous nucleotides (e.g., about 5 contiguous nucleotides to about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 6 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 8 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, or about 10 contiguous nucleotides (inclusive); about 12 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 25 contiguous nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 25 contiguous nucleotides, about 24 contiguous nucleotides, about 22 contiguous nucleotides, about 20 contiguous nucleotides, or about 18 contiguous nucleotides (inclusive); about 18 contiguous nucleotides to about 25 contiguous nucleotides, about 24 contiguous nucleotides, about 22 contiguous nucleotides, or about 20 contiguous nucleotides (inclusive); about 20 contiguous nucleotides to about 25 contiguous nucleotides, about 24 contiguous nucleotides, or about 22 contiguous nucleotides (inclusive); about 22 contiguous nucleotides to about 25 contiguous nucleotides, or about 24 contiguous nucleotides (inclusive); or about 23 contiguous nucleotides to about 25 contiguous nucleotides (inclusive)) that is not complementary to a sequence in the genomic DNA; and amplification of the genomic DNA using the two or more pairs of pre-amplification forward and reverse primers to generate about 100 to about 500 nucleotide amplification product(s).

In some embodiments, a pre-amplification step includes a locus-specific PCR reaction. In some embodiments, a locus-specific PCR reaction occurs before a locus-specific primer extension reaction (iPLEX assay).

A tag sequence can be, e.g., any contiguous sequence that is not present in the human genome. The amplification can be performed using any PCR-based assay (e.g., any of the PCR-based assays described herein or known in the art). Any of the amplification methods described herein can further include a step of sequencing the products or genotyping a target SNP present in each product (e.g., using any of the SNP genotyping assays described herein or known in the art). In some embodiments of any of the methods described herein, the tag sequence can be CAAGATGCTACGCTTC AGTC (SEQ ID NO: 1).

Some embodiments, the pre-amplification step further comprises amplification of the 100- to 500-nucleotide (e.g., 100-nucleotide to 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, 220-nucleotide, 200-nucleotide, 180-nucleotide, 160-nucleotide, 140-nucleotide, or 120-nucleotide; about 120-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, 220-nucleotide, 200-nucleotide, 180-nucleotide, 160-nucleotide, or 140-nucleotide; about 140-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, 220-nucleotide, 200-nucleotide, 180-nucleotide, or 160-nucleotide (inclusive); about 160-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, 220-nucleotide, 200-nucleotide, or 180-nucleotide (inclusive); 180-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, 220-nucleotide, or 200-nucleotide (inclusive); 200-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, 240-nucleotide, or 220-nucleotide (inclusive); 220-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, 260-nucleotide, or 240-nucleotide (inclusive); 240-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, 280-nucleotide, or 260-nucleotide (inclusive); 260-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, 300-nucleotide, or 280-nucleotide (inclusive); 280-nucleotide to 500-nucleotide, 480-nucleotide, 460-nucleotide, 440-nucleotide, 420-nucleotide, 400-nucleotide, 380-nucleotide, 360-nucleotide, 340-nucleotide, 320-nucleotide, or 300-nucleotide (inclusive); 300-nucleotide to 500-nucleotide, 480-nucleotide, 460-nucleotide, 440-nucleotide, 420-nucleotide, 400-nucleotide, 380-nucleotide, 360-nucleotide, 340-nucleotide, or 320-nucleotide (inclusive); 320-nucleotide to 500-nucleotide, 480-nucleotide, 460-nucleotide, 440-nucleotide, 420-nucleotide, 400-nucleotide, 380-nucleotide, 360-nucleotide, or 340-nucleotide (inclusive); 340-nucleotide to 500-nucleotide, 480-nucleotide, 460-nucleotide, 440-nucleotide, 420-nucleotide, 400-nucleotide, 380-nucleotide, or 360-nucleotide (inclusive); 360-nucleotide to 500-nucleotide, 480-nucleotide, 460-nucleotide, 440-nucleotide, 420-nucleotide, 400-nucleotide, or 380-nucleotide (inclusive); 380-nucleotide to 500-nucleotide, 480-nucleotide, 460-nucleotide, 440-nucleotide, 420-nucleotide, or 400-nucleotide (inclusive); 400-nucleotide to 500-nucleotide, 480-nucleotide, 460-nucleotide, 440-nucleotide, or 420-nucleotide (inclusive); 420-nucleotide to 500-nucleotide, 480-nucleotide, 460-nucleotide, or 440-nucleotide (inclusive); 440-nucleotide to 500-nucleotide, 480-nucleotide, or 460-nucleotide (inclusive); 460-nucleotide to 500-nucleotide or 480-nucleotide (inclusive); or 480-nucleotide to 500-nucleotide) amplification product(s) using a primer that comprises a sequence of about 5 contiguous nucleotides to 25 contiguous nucleotides (e.g., about 5 contiguous nucleotides to about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 6 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 8 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, or about 10 contiguous nucleotides (inclusive); about 12 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 25 contiguous nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 25 contiguous nucleotides, about 24 contiguous nucleotides, about 22 contiguous nucleotides, about 20 contiguous nucleotides, or about 18 contiguous nucleotides (inclusive); about 18 contiguous nucleotides to about 25 contiguous nucleotides, about 24 contiguous nucleotides, about 22 contiguous nucleotides, or about 20 contiguous nucleotides (inclusive); about 20 contiguous nucleotides to about 25 contiguous nucleotides, about 24 contiguous nucleotides, or about 22 contiguous nucleotides (inclusive); about 22 contiguous nucleotides to about 25 contiguous nucleotides, or about 24 contiguous nucleotides (inclusive); or about 23 contiguous nucleotides to about 25 contiguous nucleotides (inclusive)) complementary to the tag sequence.

For example, in some embodiments, a pre-amplification step can include the use of two or more (e.g., two, three, four, five, six, seven, eight, nine, ten, eleven, twelve, thirteen, or fourteen) pairs of pre-amplification reverse and forward primers selected from the group of: CAAGATGCTACGCTTCAGTCAGGAGACTATGAGGTGTGTCTCT (SEQ ID NO: 2) and CAAGATGCTACGCTTCAGTCCCTGTGCACCTCGATTGAA (SEQ ID NO: 3), respectively (target SNP of rs13182993); CAAGATGCTACGCTTCAGTCCCAAGGGGAATCACACCTC (SEQ ID NO: 4) and CAAGATGCTACGCTTCAGTCTCTGTGGAAGCATGCCACTC (SEQ ID NO: 5), respectively (target SNP of rs560681); CAAGATGCTACGCTTCAGTCTGCTGAGCCACTCTTTCAGG (SEQ ID NO: 6) and CAAGATGCTACGCTTCAGTCTTCCGGGATGTCCCGTCTTA (SEQ ID NO: 7), respectively (target SNP of rs740598); CAAGATGCTACGCTTCAGTCACAGGCAAAGAGGAACATACAGT (SEQ ID NO: 8) and CAAGATGCTACGCTTCAGTCTGCTGGCAGTGTTATTTCTTTCTC (SEQ ID NO: 9), respectively (target SNP of rs1358856); CAAGATGCTACGCTTCAGTCCTCGTTGTTCCTCTGGG (SEQ ID NO: 10) and CAAGATGCTACGCTTCAGTCCTGTTCAAGGGAAGCCTGT (SEQ ID NO: 11), respectively (target SNP of rs9951171); CAAGATGCTACGCTTCAGTCGGATCAGGAAACAGGGAGCC (SEQ ID NO: 12) and CAAGATGCTACGCTTCAGTCGAAGACTCTGTCCCAGCCAC (SEQ ID NO: 13), respectively (target SNP of rs7520386); CAAGATGCTACGCTTCAGTCGCTTCTTCTGCCACATCCCT (SEQ ID NO: 14) and CAAGATGCTACGCTTCAGTCGCATTTTCATGGAGGGCCAC (SEQ ID NO: 15), respectively (target SNP of rs13218440); CAAGATGCTACGCTTCAGTCTTGCCATGTTTGTCACAGGT (SEQ ID NO: 16) and CAAGATGCTACGCTTCAGTCACCTTGGTTTCTTGATTATGTTGAT (SEQ ID NO: 17), respectively (target SNP of rs279844); CAAGATGCTACGCTTCAGTCTGAATCCTCCCCCAAGCTG (SEQ ID NO: 18) and CAAGATGCTACGCTTCAGTCTTCTCCTCTTCCTGGGCTGA (SEQ ID NO: 19), respectively (target SNP of rs1058083); CAAGATGCTACGCTTCAGTCGCACATTAAATGGGTTCCAG (SEQ ID NO: 20) and CAAGATGCTACGCTTCAGTCGAAATACGAAGGACACAAAACCTC (SEQ ID NO: 21) (target SNP of rs2032597), respectively; CAAGATGCTACGCTTCAGTCTGTTGCTGGCAAGACACTTC (SEQ ID NO: 22) and CAAGATGCTACGCTTCAGTCTGCCTTTGCTACAACTCTCCT (SEQ ID NO: 23), respectively (target SNP of rs2032631); CAAGATGCTACGCTTCAGTCTAGCCCCACGTCACTTCAG (SEQ ID NO: 24) and CAAGATGCTACGCTTCAGTCTTCTTGGAAGGTGGTCCTGG (SEQ ID NO: 25), respectively (target SNP of rs2272998); CAAGATGCTACGCTTCAGTCAGGACCTGTAAGAGTCTGTGATT (SEQ ID NO: 26) and CAAGATGCTACGCTTCAGTCTGTATCCCAGGTTCAATGACTGT (SEQ ID NO: 27), respectively (target SNP of rs12997453); CAAGATGCTACGCTTCAGTCACACCCTTACCTGTATTTTCTGA (SEQ ID NO: 28) and CAAGATGCTACGCTTCAGTCGTGCACATTCTAAGAACTGGTGAT (SEQ ID NO: 29), respectively (target SNP of rs214955); CAAGATGCTACGCTTCAGTCTGCTTACAGTGATTCTTGCCT (SEQ ID NO: 30) and CAAGATGCTACGCTTCAGTCAGTCTTTTGCACCAAGTCTTTTT (SEQ ID NO: 31), respectively (target SNP of rs13134862); and CAAGATGCTACGCTTCAGTCGGAAGATGCTTGAACTCCCCA (SEQ ID NO: 32) and CAAGATGCTACGCTTCAGTCACATCAAAGCTGGGAACCG (SEQ ID NO: 33), respectively (target SNP of rs1410059).

Some embodiments where a pre-amplification step is performed, the method can further include a step of removing unincorporated deoxynucleotides (dNTPs) prior to elongating the two or more oligonucleotide to generate the set of two or more SNP amplification products. In some embodiments, an alkaline phosphatase (e.g., shrimp alkaline phosphatase (SAP)) is used to remove unincorporated dNTPs. Methods of using an alkaline phosphatase (e.g., SAP) are well known in the art, and include adding the alkaline phosphatase after the pre-amplification step, incubating the mixture containing the alkaline phosphatase at 37° C. for about 10 minutes to about 120 minutes (e.g., about 10 minutes to about 110 minutes, about 100 minutes, about 90 minutes, about 80 minutes, about 70 minutes, about 60 minutes, about 50 minutes, about 40 minutes, about 30 minutes, or about 20 minutes (inclusive); about 20 minutes to about 120 minutes, about 110 minutes, about 100 minutes, about 90 minutes, about 80 minutes, about 70 minutes, about 60 minutes, about 50 minutes, about 40 minutes, about 30 minutes, or about 20 minutes (inclusive); about 20 minutes to about 120 minutes, about 110 minutes, about 100 minutes, about 90 minutes, about 80 minutes, about 70 minutes, about 60 minutes, about 50 minutes, about 40 minutes, or about 30 minutes (inclusive); about 30 minutes to about 120 minutes, about 110 minutes, about 100 minutes, about 90 minutes, about 80 minutes, about 70 minutes, about 60 minutes, about 50 minutes, or about 40 minutes (inclusive); about 40 minutes to about 120 minutes, about 110 minutes, about 100 minutes, about 90 minutes, about 80 minutes, about 70 minutes, about 60 minutes, or about 50 minutes (inclusive); about 50 minutes to about 120 minutes, about 110 minutes, about 100 minutes, about 90 minutes, about 80 minutes, about 70 minutes, or about 60 minutes (inclusive); about 60 minutes to about 120 minutes, about 110 minutes, about 100 minutes, about 90 minutes, about 80 minutes, or about 70 minutes (inclusive); about 70 minutes to about 120 minutes, about 110 minutes, about 100 minutes, about 90 minutes, or about 80 minutes (inclusive); about 80 minutes to about 120 minutes, about 110 minutes, about 100 minutes, or about 90 minutes (inclusive); about 90 minutes to about 120 minutes, about 110 minutes, or about 100 minutes (inclusive); about 100 minutes to about 120 minute or about 110 minutes (inclusive); or about 110 minutes to about 120 minutes (inclusive)), and then incubating the mixture containing SAP at 85° C. for about 5 to about 60 minutes (e.g., about 5 minutes to about 50 minutes, about 40 minutes, about 30 minutes, about 20 minutes, or about 10 minutes (inclusive); about 10 minutes to about 60 minutes, about 50 minutes, about 40 minutes, about 30 minutes, or about 20 minutes (inclusive); about 20 minutes to about 60 minutes, about 50 minutes, about 40 minutes, or about 30 minutes (inclusive); about 30 minutes to about 60 minutes, about 50 minutes, or about 40 minutes (inclusive); about 40 minutes to about 60 minutes or about 50 minutes (inclusive); or about 50 minutes to about 60 minutes (inclusive)) to inactivate SAP enzyme. In some embodiments of any of the methods described herein, the SAP treated sample can be stored, e.g., for at least 1 hour (e.g., at least 6 hours, at least 12 hours, at least 1 day, at least 2 days, at least 3 days, at least 4 days, at least 5 days, at least 6 days, or at least 7 days) at a temperature below 25° C. (e.g., at about 15° C., at about 10° C., at about 4° C., at about 0° C., at about −20° C., at about −40° C., at about −80° C., at about −86° C., or at about −196° C.).

An assay to determine the mass distribution of a set of SNP amplification products in a urine sample and/or an assay to determine the control mass distribution (described below) can be performed at the same time, substantially the same time, or during an overlapping time period as one or more of: an assay to determine the presence of one or more saliva proteins in the urine sample (e.g., using an aliquot of the same urine sample), an assay to determine the level(s) of one or more drugs and/or one or more drug metabolites in the urine sample (e.g., using an aliquot of the same urine sample), and an assay to determine the genotype of at least one target SNP in the isolated genomic DNA test sample or the control sample (e.g., using an aliquot of the same urine sample) is performed.

### Methods of Determining a Mass Distribution

Exemplary methods for determining a mass distribution of a set of SNP amplification products (e.g., any of the sets of SNP amplification products described herein) and/or a control mass distribution are described herein. Additional methods for determining a mass distribution of a set of SNP amplification products (e.g., any of the sets of SNP amplification products described herein) and/or a control mass distribution are known in the art.

In some embodiments, the mass distribution of the set of SNP amplification products and/or the control mass distribution is/are determined using mass spectrometry (e.g., matrix-assisted laser desorption/ionization time-of-flight (MALDI-TOF) analysis, tandem mass spectrometry analysis, gas chromatography-mass spectrometry analysis, capillary electrophoresis-mass spectrometry analysis, and ion mobility spectrometry-mass spectrometry analysis). In some embodiments, the mass distribution of the set of SNP amplification products and/or the control mass distribution is determined after elongation of two or more oligonucleotide probes is performed using a locus-specific PCR reaction. In some embodiments, the locus-specific PCR reaction is a locus-specific primer extension reaction (iPLEX assay).

In some embodiments, the methods provided herein include placing the set of SNP amplification products in or onto a solid support that can be placed into a mass spectrometer. In some embodiments, the mass spectrometer is a MALDI-TOF mass spectrometer. In some embodiments, the solid support is a multi-well assay plate, a functionalized plate, or a chip. Additional aspects and examples of solid supports are known in the art.

Methods of using a mass spectrometer, e.g., a MALDI-TOF mass spectrometer, sample preparation, and common matrices used, are well known in the art. One skilled in the art will appreciate that additional steps can be added to the methods described herein and other materials can be used to perform any of the steps of the methods described herein. See, e.g., Nakorchevsky et al., Journal of Blood Disorders & Transfusion 6:340, 2016; Erhich et al., PNAS 102(44): 15785-15790, 2005; Gabriel et al., Current Protocols in Human Genetics 2009, Chapter 2: Unit 2.12; Wang et al., Philos. Trans. A Math Phys. Eng. Sci. 374:2079, 2016; Rahi et al., Front. Microbiol. 7:1359, 2016; Karger, Proteomics Clin. Appl. 10:982-993, 2016; Charretier et al., Proteomics Clin. Appl. 10:964-981, 2016; Hajduk et al., Clin. Chim. Acta 458:84-98, 2016; Duncan et al., Clin. Chem. 62:134-143, 2016; Cho et al., Adv. Clin. Chem. 69:209-254, 2015; Kriegsmann et al., Int. J. Oncol. 46:893-906, 2015; Nomura, Biochim. Biophys. Acta 1854:528-537, 2015; and Singhal et al., Front. Microbiol. 6:791, 2015.

Generally, in MALFI-TOF analysis, a sample is embedded in a matrix and a laser beam is used as a source of desorption and ionization. After the set of SNP amplification products and/or control sample is vaporized and ionized, respectively, the set of SNP amplification products and/or control sample is transferred electrostatically into a time-of-flight mass spectrometer (TOF-MS). The time of ion flight differs according to the mass-to-charge ratio (m/z) value of each ion (e.g., ions with smaller m/z value (lighter ions) and more highly charged ions move faster). The result of a MALDI-TOF assay is a collection of m/z ratios and intensity values. Each m/z ratio has an intensity value that is proportional to the amount of hits that were detected, which in turn is proportional to the amount of particles with that mass in the sample (i.e., the number or amount of a specific SNP amplification product in the set of SNP amplification products).

In some embodiments, the determining step (g) further includes a standardization or normalization step, where the mass distribution of the set of SNP amplification products and/or the control mass distribution are normalized to facilitate comparison (e.g., the series of intensity values of the set of SNP amplification products are normalized to the series of intensity values of the control sample). Non-limiting examples of normalization techniques include: cumulative intensity, linear regression, LOWESS, Quantile, Median, internal standard, standard deviation of noise, total ion current (or total area under the curve) and top-L. See, e.g., Callister et al., Journal of Proteom Research 5:277-286, 2006; Ballman et al., Bioinformatics 20(16): 2778-2786, 2004; and Wang et al., Pacific Symposium on Biocomputing 11:315-326, 2006; Griffin et al., Nature Biotechnology 28:83-89, 2010, Nakorchevsky et al., Journal of Blood Disorders & Transfusion 6:340, 2016; and Deininger et al., Anal. Bioanal. Chem 401(1): 167-181, 2011.

In some embodiments, where the elongation step fails to produce a SNP amplification product using a specific oligonucleotide probe, an unextended primer peak is observed in the MALDI-TOF analysis spectra (corresponding to the un-elongated specific oligonucleotide probe).

Some embodiments of any of these methods can further include characterizing a urine sample through the use of a clustering algorithm (e.g., a hierarchical clustering algorithm, a k-means clustering algorithm, or a statistical distribution model). Non-limiting examples of clustering algorithms are described herein. Additional examples of clustering algorithms are known in the art.

Some embodiments of any of these methods can further include further characterizing a urine sample by performing regression analysis on the values of the one or more principle component(s). Some embodiments of these methods can further include comparing the determined mass distribution of the urine sample to a mass distribution of an additional urine sample obtained from the subject.

In some embodiments, the control mass distribution is in the subject's clinical record (e.g., provided as a computer readable medium). In some embodiments, where the mass distribution of the set of SNP amplification products is about or at least about 90.0% (e.g., about or at least about 90.1%, about or at least about 90.2%, about or at least about 90.3%, about or at least about 90.4%, about or at least about 90.5%, about or at least about 90.6%, about or at least about 90.7%, about or at least about 90.8%, about or at least about 90.9%, about or at least about 91.0%, about or at least about 91.1%, about or at least about 91.2%, about or at least about 91.3%, about or at least about 91.4%, about or at least about 91.5%, about or at least about 91.6%, about or at least about 91.7%, about or at least about 91.8%, about or at least about 91.9%, about or at least about 92.0%, about or at least about 92.1%, about or at least about 92.2%, about or at least about 92.3%, about or at least about 92.4%, about or at least about 92.5%, about or at least about 92.6%, about or at least about 92.7%, about or at least about 92.8%, about or at least about 92.9%, about or at least about 93.0%, about or at least about 93.1%, about or at least about 93.2%, about or at least about 93.3%, about or at least about 93.4%, about or at least about 93.5%, about or at least about 93.6%, about or at least about 93.7%, about or at least about 93.8%, about or at least about 93.9%, about or at least about 94.0%, about or at least about 94.1%, about or at least about 94.2%, about or at least about 94.3%, about or at least about 94.4%, about or at least about 94.5%, about or at least about 94.6%, about or at least about 94.7%, about or at least about 94.8%, about or at least about 94.9%, about or at least about 95.0%, about or at least about 95.1%, about or at least about 95.2%, about or at least about 95.3%, about or at least about 95.4%, about or at least about 95.5%, about or at least about 95.6%, about or at least about 95.7%, about or at least about 95.8%, about or at least about 95.9%, about or at least about 96.0%, about or at least about 96.1%, about or at least about 96.2%, about or at least about 96.3%, about or at least about 96.4%, about or at least about 96.5%, about or at least about 96.6%, about or at least about 96.7%, about or at least about 96.8%, about or at least about 96.9%, about or at least about 97.0%, about or at least about 97.1%, about or at least about 97.2%, about or at least about 97.3%, about or at least about 97.4%, about or at least about 97.5%, about or at least about 97.6%, about or at least about 97.7%, about or at least about 97.8%, about or at least about 97.9%, about or at least about 98.0%, about or at least about 98.1%, about or at least about 98.2%, about or at least about 98.3%, about or at least about 98.4%, about or at least about 98.5%, about or at least about 98.6%, about or at least about 98.7%, about or at least about 98.8%, about or at least about 98.9%, about or at least about 99.0%, about or at least about 99.1%, about or at least about 99.2%, about or at least about 99.3%, about or at least about 99.4%, about or at least about 99.5%, about or at least about 99.6%, about or at least about 99.7%, about or at least about 99.8%, or about or at least about 99.9%) identical to the control mass distribution, a urine sample is identified as originating from the subject.

In some embodiments, the mass distribution of the set of SNP amplification products that is about or less than about 99.5% (e.g., about or less than about 99.4%, about or less than about 99.3%, about or less than about 99.2%, about or less than about 99.1%, about or less than about 99.0%, about or less than about 98.9%, about or less than about 98.8%, about or less than about 98.7%, about or less than about 98.6%, about or less than about 98.5%, about or less than about 98.4%, about or less than about 98.3%, about or less than about 98.2%, about or less than about 98.1%, about or less than about 98.0%, about or less than about 97.9%, about or less than about 97.8%, about or less than about 97.7%, about or less than about 97.6%, about or less than about 97.5%, about or less than about 97.4%, about or less than about 97.3%, about or less than about 97.2%, about or less than about 97.1%, about or less than about 97.0%, about or less than about 96.9%, about or less than about 96.8%, about or less than about 96.7%, about or less than about 96.6%, about or less than about 96.5%, about or less than about 96.4%, about or less than about 96.3%, about or less than about 96.2%, about or less than about 96.1%, about or less than about 96.0%, about or less than about 95.9%, about or less than about 95.8%, about or less than about 95.7%, about or less than about 95.6%, about or less than about 95.5%, about or less than about 95.4%, about or less than about 95.3%, about or less than about 95.2%, about or less than about 95.1%, about or less than about 95.0%, about or less than about 94.9%, about or less than about 94.8%, about or less than about 94.7%, about or less than about 94.6%, about or less than about 94.5%, about or less than about 94.4%, about or less than about 94.3%, about or less than about 94.2%, about or less than about 94.1%, about or less than about 94.0%, about or less than about 93.9%, about or less than about 93.8%, about or less than about 93.7%, about or less than about 93.6%, about or less than about 93.5%, about or less than about 93.4%, about or less than about 93.3%, about or less than about 93.2%, about or less than about 93.1%, about or less than about 93.0%, about or less than about 92.9%, about or less than about 92.8%, about or less than about 92.7%, about or less than about 92.6%, about or less than about 92.5%, about or less than about 92.4%, about or less than about 92.3%, about or less than about 92.2%, about or less than about 92.1%, about or less than about 92.0%, about or less than about 91.9%, about or less than about 91.8%, about or less than about 91.7%, about or less than about 91.6%, about or less than about 91.5%, about or less than about 91.4%, about or less than about 91.3%, about or less than about 91.2%, about or less than about 91.1, about or less than about 91.0%, about or less than about 90.0%, about or less than about 90.9%, about or less than about 90.8%, about or less than about 90.7%, about or less than about 90.6%, about or less than about 90.5%, about or less than about 90.4%, about or less than about 90.3%, about or less than about 90.2%, about or less than about 90.1%, about or less than about 90.0%, about or less than about 89.5%, about or less than about 89.0%, about or less than about 88.5%, about or less than about 88.0%, about or less than about 87.5%, about or less than about 87.0%, about or less than about 86.5%, about or less than about 86.0%, about or less than about 85.5%, about or less than about 85.0%, about or less than about 84.5%, about or less than about 84.0%, about or less than about 83.5%, about or less than about 83.0%, about or less than about 82.5%, about or less than about 82.0%, about or less than about 81.5%, about or less than about 81.0%, about or less than about 80.5%, about or less than about 80.0%, about or less than about 78%, about or less than about 76%, about or less than about 74%, about or less than about 72%, about or less than about 70%, about or less than about 68%, about or less than about 66%, about or less than about 64%, about or less than about 62%, about or less than about 60%, about or less than about 58%, about or less than about 56%, about or less than 54%, about or less than about 52%, about or less than about 50%, about or less than about 48%, about or less than about 46%, about or less than about 44%, about or less than about 42%, about or less than about 40%, about or less than about 38%, about or less than about 36%, about or less than about 34%, about or less than about 32%, about or less than about 30%, about or less than about 28%, about or less than about 26%, about or less than about 24%, about or less than about 22%, or about or less than about 20%) identical to the control mass distribution, a urine sample is identified as not originating from the subject and/or being adulterated.

### Control Mass Distribution

The methods described herein further include comparing the mass distribution of a set of SNP amplification products to a control mass distribution (e.g., a control mass distribution determined from a buccal cell sample obtained from the subject).

A control mass distribution can be obtained from a buccal cell sample, a hair sample, a blood sample, a plasma sample, a serum sample, or a witnessed urine sample from the subject. Some of the methods described herein further include a step of obtaining a buccal cell sample, a hair sample, a blood sample, a plasma sample, a serum sample, or a witnessed urine sample from the subject. Some embodiments of the methods provided herein further include performing an assay to determine the mass distribution of the buccal cell sample, a hair sample, a blood sample, a plasma sample, a serum sample, or a witnessed urine sample from the subject (i.e., the control mass distribution).

A control mass distribution can be determined by: (i) obtaining a buccal cell sample, a hair sample, a blood sample, a plasma sample, a serum sample, a witnessed urine sample from the subject; (ii) enriching the sample in (i) for mammalian cells, if present; (iii) isolating any genomic DNA from the enriched sample of step (ii) to form an isolated genomic DNA control sample; (iv) contacting a set of two or more oligonucleotide probes (e.g., any of the sets of two or more oligonucleotide probes described herein) to the genomic DNA in the isolated genomic DNA control sample in (iii), where each of the two or more oligonucleotide probes hybridizes to a sequence of genomic DNA that is one nucleotide(s) upstream of a different target single nucleotide polymorphism (SNP); (v) elongating the two or more oligonucleotide probes hybridized to the genomic DNA by one nucleotide using chain-terminating nucleotide(s) to generate a set of two or more SNP amplification products (e.g., any of the two or more SNP amplification products described herein); and (vi) determining the mass distribution of the set of SNP amplification products.

The step of enriching the buccal cell sample for mammalian cells, if present, can be performed using any of the exemplary methods for performing such enrichment described herein or known in the art. The step of isolating any genomic DNA from the enriched sample of step (ii) can be performed using any methods for isolating genomic DNA from an enriched sample described herein or known in the art. The step of contacting a set of two or more oligonucleotide probes to the genomic DNA in the isolated genomic DNA control sample can be performed using any of the exemplary methods described herein or known in the art. The step of elongating the two or more oligonucleotide probes hybridized to the genomic DNA by one nucleotide using chain-terminating nucleotide(s) can be performed using any of the methods described herein or known in the art. In some embodiments, the elongation includes the use of a PCR assay (e.g., a locus-specific primer extension reaction (iPLEX assay), or a real-time PCR assay). In some embodiments, prior to elongation, the method can include a pre-amplification step (e.g., any of the pre-amplification steps described herein or known in the art). Some embodiments that further include a pre-amplification step can further include a step of removing any unincorporated dNTPs (i.e., after the pre-amplification step but before the elongation step (e.g., using any of the methods for removing any unincorporated dNTPs described herein or known in the art)). One skilled in the art will appreciate that additional steps can be added to the methods described herein and other materials can be used to perform any of the steps of the methods described herein.

In some examples, the methods used to generate the set of SNP amplification products and determine the mass distribution of the set of SNP amplification products of the urine sample from the subject are the same or substantially the same as the methods used to generate the set of SNP amplification products and determine the mass distribution of the set of SNP amplification products of the buccal cell sample, the hair sample, the blood sample, the plasma sample, the serum sample, or the witnessed urine sample from the subject.

### Methods of Matching a Urine Sample to a Subject

Provided herein are methods of matching a urine sample to a subject (e.g., any of the subjects described herein) that include: (a) providing a urine sample (e.g., any of the urine samples described herein) from a subject (e.g., any of the subjects described herein, e.g., a human); (b) enriching the urine sample for mammalian cells, if present; (c) isolating any genomic DNA from the enriched sample of step (b) to form an isolated genomic DNA test sample; (d) contacting a set of two or more oligonucleotide probes to the genomic DNA in the isolated genomic DNA test sample, wherein each of the two or more oligonucleotide probes hybridizes to a sequence of genomic DNA that is one nucleotide upstream of a different target single nucleotide polymorphism (SNP); (e) elongating the two or more oligonucleotide probes hybridized to the genomic DNA by one nucleotide using chain-terminating nucleotide(s) to generate a set of two or more SNP amplification products; (f) determining the mass distribution of the set of SNP amplification products; (g) comparing the determined mass distribution of the set of SNP amplification products to a control mass distribution; and (h) identifying a urine sample having a mass distribution that is the same as the control mass distribution as originating from the subject; or identifying a urine sample having a mass distribution that is not the same as the control mass distribution as not originating from the subject.

The subject can be any of the subjects described herein. The urine sample can be any of the urine samples described herein.

The step of enriching the urine sample for mammalian cells, if present, can be performed using any of the exemplary methods for performing such enrichment described herein or known in the art. The step of isolating any genomic DNA from the enriched sample of step (b) can be performed using any methods for isolating genomic DNA from an isolated genomic DNA test sample described herein or known in the art.

The step of contacting a set of two or more oligonucleotide probes (e.g., any of the exemplary sets of two or more oligonucleotides probes described herein) to the genomic DNA in the isolated genomic DNA test sample can be performed using any of the exemplary methods described herein or known in the art. In some embodiments, each of the two or more oligonucleotide probes hybridizes to a sequence of genomic DNA that is two nucleotides upstream of a different target single nucleotide polymorphism (SNP); and step (e) includes elongating the two or more oligonucleotide probes hybridized to the genomic DNA by two nucleotides using chain-terminating nucleotide(s) to generate the set of two or more SNP amplification products. In some embodiments, each of the two or more oligonucleotide probes hybridizes to a sequence of genomic DNA that is three nucleotides upstream of a different target single nucleotide polymorphism (SNP); and step (e) includes elongating the two or more oligonucleotide probes hybridized to the genomic DNA by three nucleotides using chain-terminating nucleotide(s) to generate the set of two or more SNP amplification products. In some embodiments, each of the two or more oligonucleotide probes hybridizes to a sequence of genomic DNA that is four nucleotides upstream of a different target single nucleotide polymorphism (SNP); and step (e) includes elongating the two or more oligonucleotide probes hybridized to the genomic DNA by four nucleotides using chain-terminating nucleotide(s) to generate the set of two or more SNP amplification products. In some embodiments, each of the two or more oligonucleotide probes hybridizes to a sequence of genomic DNA that is five nucleotides upstream of a different target single nucleotide polymorphism (SNP); and step (e) includes elongating the two or more oligonucleotide probes hybridized to the genomic DNA by five nucleotides using chain-terminating nucleotide(s) to generate the set of two or more SNP amplification products. In some embodiments, each of the two or more oligonucleotide probes hybridizes to a sequence of genomic DNA that is six nucleotides upstream of a different target single nucleotide polymorphism (SNP); and step (e) includes elongating the two or more oligonucleotide probes hybridized to the genomic DNA by six nucleotides using chain-terminating nucleotide(s) to generate the set of two or more SNP amplification products. In some embodiments, each of the two or more oligonucleotide probes hybridizes to a sequence of genomic DNA that is seven nucleotides upstream of a different target single nucleotide polymorphism (SNP); and step (e) includes elongating the two or more oligonucleotide probes hybridized to the genomic DNA by seven nucleotides using chain-terminating nucleotide(s) to generate the set of two or more SNP amplification products. In some embodiments, each of the two or more oligonucleotide probes hybridizes to a sequence of genomic DNA that is eight nucleotides upstream of a different target single nucleotide polymorphism (SNP); and step (e) includes elongating the two or more oligonucleotide probes hybridized to the genomic DNA by eight nucleotides using chain-terminating nucleotide(s) to generate the set of two or more SNP amplification products. In some embodiments, each of the two or more oligonucleotide probes hybridizes to a sequence of genomic DNA that is nine nucleotides upstream of a different target single nucleotide polymorphism (SNP); and step (e) includes elongating the two or more oligonucleotide probes hybridized to the genomic DNA by nine nucleotides using chain-terminating nucleotide(s) to generate the set of two or more SNP amplification products. In some embodiments, each of the two or more oligonucleotide probes hybridizes to a sequence of genomic DNA that is ten nucleotides upstream of a different target single nucleotide polymorphism (SNP); and step (e) includes elongating the two or more oligonucleotide probes hybridized to the genomic DNA by ten nucleotides using chain-terminating nucleotide(s) to generate the set of two or more SNP amplification products.

The step of elongating the two or more oligonucleotide probes hybridized to the genomic DNA by one nucleotide using chain-terminating nucleotide(s) (e.g., any of the chain-terminating nucleotides described herein) can be performed using any of the methods described herein or known in the art.

In some embodiments, the determination of the mass distribution of the set of SNP amplification products in step (f) includes the use of mass spectrometry (e.g., any of the types of mass spectrometry described herein, e.g., through the use of matrix-assisted laser desorption/ionization time-of-flight (MALDI-TOF) analysis).

Some embodiments of these methods further include performing an assay to confirm the identity of at least one (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, at least 50, at least 52, at least 54, at least 56, at least 58, at least 60, at least 62, at least 64, at least 66, at least 68, at least 70, at least 72, at least 74, at least 76, at least 78, at least 80, at least 82, at least 84, at least 86, at least 88, at least 90, at least 92, at least 92, at least 94, at least 96, at least 98, or at least 100) target SNP in the isolated genomic DNA test sample of step (c). Non-limiting examples of assays for determining/confirming the identity of a target SNP are described herein. Additional methods for determining/confirming the identity of a target SNP are known in the art.

In some embodiments, the comparing step (g) is performed using a computer algorithm. In some examples, the comparing step (g) is performed by comparing the area under the curve (AUC) of the mass spectrometry spectrum.

In some embodiments, the method includes identifying a urine sample having a mass distribution that is the same or substantially the same as the control mass distribution as originating from the subject. Non-limiting methods for identifying a urine sample as originating from the subject as specified in step (h) are described herein.

In some embodiments, the method includes identifying a urine sample having a mass distribution as not originating from the subject. Non-limiting methods for identifying a urine sample as not originating from the subject as specified in step (h) are described herein.

In some embodiments, a urine sample identified as not originating from the subject comprises, consists essentially of, or consists of synthetic urine. In some embodiments, a urine sample identified as not originating from the subject comprises, consists essentially of, or consists of genomic DNA that is not of human origin.

Such embodiments can further include performing an assay to determine the level of one or more drugs and/or the level of one or more drug metabolites (e.g., any of the exemplary drugs and/or drug metabolites described herein or known in the art) in the urine sample identified in step (h) as originating from the subject.

Some methods further include: (i) performing an assay to identify the presence of one or more saliva proteins (e.g., one or more of human statherin, human alpha-amylase, and human lysozyme) in the urine sample, and (j) identifying a urine sample having a mass distribution that is the same as the control mass distribution, and a detectable level of the one or more saliva proteins (e.g., one or more of human statherin, human alpha-amylase, and human lysozyme) as being adulterated. In some embodiments, the assay in step (i) is an enzyme activity assay or an enzyme-linked immunosorbent assay (ELISA).

### Assays to Determine/Confirm the Identity of a Target SNP

Some of the methods provided herein include a step of performing an assay to determine/confirm the genotype of at least one (e.g., at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) target SNP in the isolated genomic DNA test sample and/or the control sample. In some embodiments where the genotype of at least two (e.g., at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) target SNPs are determined/confirmed, the at least two target SNPs include at least one SNP from at least two different chromosomes. In some embodiments where the genotype of at least three (e.g., at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) target SNPs are determined/confirmed, the at least three target SNPs include at least one SNP from at least three different chromosomes. In some embodiments where the genotype of at least four (e.g., at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) target SNPs are determined/confirmed, the at least four target SNPs include at least one SNP from at least four different chromosomes. In some embodiments where the genotype of at least six (e.g., at least seven, at least eight, at least nine, at least ten, at least eleven, at least twelve, at least thirteen, at least fourteen, at least fifteen, at least sixteen, at least seventeen, at least eighteen, at least nineteen, or at least twenty) target SNPs are determined/confirmed, the at least six target SNPs include at least one SNP from at least six different chromosomes. In some embodiments where the genotype of at least eight (e.g., at least nine, at least ten, at least eleven, at least twelve, at least thirteen, at least fourteen, at least fifteen, at least sixteen, at least seventeen, at least eighteen, at least nineteen, or at least twenty) target SNPs are determined/confirmed, the at least eight target SNPs include at least one target SNP from at least eight different chromosomes. In some embodiments, where the subject is a genetic male, the at least one target SNP that is determined/confirmed includes a target SNP (e.g., two SNPs) located on a Y chromosome.

In some embodiments, the genotype of the at least one target SNP (e.g., at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) that is determined/confirmed has a minor allele frequency of >0.4. In some embodiments, the genotype of at least one target SNP (e.g., two, three, four, five, six, seven, eight, nine, ten, eleven, twelve, thirteen, fourteen, fifteen, sixteen, seventeen, eighteen, or nineteen target SNPs) having a minor allele frequency of >0.4 that is determined/confirmed is selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments, the genotype of at least one target SNP (e.g., two, three, four, five, six, seven, eight, nine, ten, eleven, twelve, thirteen, or fourteen target SNPs) that is determined/confirmed is/are selected from the group of: rs7520386, rs560681, rs9951171, rs1058083, rs1358856, rs214955, rs740598, rs279844, rs13218440, rs2272998, rs12997453, rs13134862, rs13182883, and rs1410059.

A variety of assays for determining/confirming the genotype of a SNP are known in the art. Non-limiting examples of such assays (which can be used in any of the methods described herein) include: dynamic allele-specific hybridization (see, e.g., Howell et al., Nature Biotechnology. 17:87-88, 1999), molecular beacon assays (see, e.g., Marras et al., “Genotyping Single Nucleotide Polymorphisms with Molecular Beacons,” In Kwok (Ed.), Single Nucleotide Polymorphisms: Methods and Protocols, Humana Press, Inc., Totowa, N.J., Vol. 212, pp. 111-128, 2003), SNP microarrays (see, e.g., Affymetrix Human SNP 5.0 GeneChip), restriction fragment length polymorphism (RFLP) (see, e.g., Ota et al., Nature Protocols 2:2857-2864, 2007), PCR-based assays (e.g., tetraprimer ARMS-PCR (see, e.g., Zhang et al., Plos One 8:e62126, 2013), real-time PCR, allele-specific PCR (see, e.g., Gaudet et al., Methods Mol. Biol. 578:415-424, 2009), a TaqMan Assay SNP Genotyping (see, e.g., Woodward, Methods Mol. Biol. 1145:67-74, 2014, and TaqMan® OpenArray® Genotyping Plates from Life Technologies)), Flap endonuclease assays (also called Invader assays) (see, e.g., Olivier et al., Mutat. Res. 573:103-110, 2005), oligonucleotide ligation assays (see, e.g., Bruse et al., Biotechniques 45:559-571, 2008), single strand conformational polymorphism assays (see, e.g., Tahira et al., Human Mutat. 26:69-77, 2005), temperature gradient gel electrophoresis (see, e.g., Jones et al., “Temporal Temperature Gradient Electrophoresis for Detection of Single Nucleotide Polymorphisms,” in Single Nucleotide Polymophisms: Methods and Protocols, Volume 578, pp. 153-165, 2008) or temperature gradient capillary electrophoresis, denaturing high performance liquid chromatography (see, e.g., Yu et al., J. Clin. Pathol. 58:479-485, 2005), high-resolution melting of an amplified sequence containing the SNP (see, e.g., Wittwer et al., Clinical Chemistry 49:853-860, 2003), or sequencing (e.g., Maxam-Gilbert sequencing, chain-termination methods, shotgun sequencing, bridge PCR, and next-generation sequencing methods (e.g., massively parallel signature sequencing, polony sequencing, 454 pyrosequencing, Illumina (Solexa) sequencing, SOLiD sequencing, Ion Torrent semiconductor sequence, DNA nanoball sequencing, heliscope single molecule sequencing, and single molecule real-time sequencing), a locus-specific primer extension reaction (iPLEX assay) (see, e.g., Gabriel et al., Current Protocols in Human Genetics 2009, Chapter 2: Unit 2.12). Additional details and a summary of various next-generation sequencing methods are described in Koboldt et al., Cell 155:27-38, 2013.

In some embodiments of any of the methods described herein, the genotyping of the at least one target SNP (e.g., at least 6 target SNPs) includes a PCR assay (e.g., a real-time PCR-assay, e.g., a real-time PCR-based SNP genotyping assay) (with or without a prior pre-amplification step (e.g., any of the pre-amplification methods described herein)). In some embodiments of any of the methods described herein the genotyping of the at least one target SNP (e.g., at least 6 target SNPs) is performed using TaqMan®-based sequencing (e.g., TaqMan®-based OpenArray® sequencing, e.g., high throughput TaqMan®-based Open Array® sequencing) (with or without a prior pre-amplification step (e.g., any of the pre-amplification methods described herein)).

Methods for designing primers for use in the various target SNP genotyping assays described herein are well known in the art. For example, several vendors provide free software for designing forward and reverse primers for use in any of the target SNP genotyping assays described herein. A forward or reverse primer for use in any of the target SNP genotyping assays described herein can contain at least 10 (e.g., 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides). In some embodiments, a forward or reverse primer used in any of the target SNP genotyping assays described herein can include a label (e.g., any of the exemplary labels described herein) or can include a contiguous tag sequence (e.g., between about 5 nucleotides and about 25 nucleotides, between about 10 nucleotides and about 25 nucleotides, between about 10 nucleotides and 20 nucleotides, between about 5 nucleotides and about 20 nucleotides) that does not hybridize to a sequence within the subject's genome (e.g., the human genome).

Non-limiting exemplary pairs of forward and reverse primers that can be used in a genotyping assay include: SEQ ID NO: 2 and SEQ ID NO: 3, respectively, to amplify a sequence containing rs13182883; SEQ ID NO: 4 and SEQ ID NO: 5, respectively, to amplify a sequence containing rs560681; SEQ ID NO: 6 and SEQ ID NO: 7, respectively, to amplify rs740598; SEQ ID NO: 8 and SEQ ID NO: 9, respectively, to amplify a sequence containing rs1358856; SEQ ID NO: 10 and SEQ ID NO: 11, respectively, to amplify a sequence containing rs9951171; SEQ ID NO: 12 and SEQ ID NO: 13, respectively, to amplify a sequence containing rs5720386; SEQ ID NO: 14 and SEQ ID NO: 15, respectively, to amplify a sequence containing rs13218440; SEQ ID NO: 16 and SEQ ID NO: 17, respectively, to amplify a sequence containing rs279844; SEQ ID NO: 18 and SEQ ID NO: 19, respectively, to amplify a sequence containing rs1058083; SEQ ID NO: 20 and SEQ ID NO: 21, respectively, to amplify a sequence containing rs2032597; SEQ ID NO: 22 and SEQ ID NO: 23, respectively, to amplify a sequence containing rs2032631; SEQ ID NO: 24 and SEQ ID NO: 25, respectively, to amplify a sequence containing rs2272998; SEQ ID NO: 26 and SEQ ID NO: 27, respectively, to amplify a sequence containing rs12997453; SEQ ID NO: 28 and SEQ ID NO: 29, respectively, to amplify a sequence containing rs214955; SEQ ID NO: 30 and SEQ ID NO: 31, respectively, to amplify a sequence containing rs13134862; and SEQ ID NO: 32 and SEQ ID NO: 33, respectively to amplify a sequence containing rs1410059. The sequence surrounding each SNP described herein (or any SNP genotyped in the methods described herein) can be found using the database of human SNPs (dbSNP) on the NCBI website (ncbi.nlm.nih.gov/projects/SNP/).

Any of the target SNP genotyping assays described herein can include a pre-amplification step (e.g., any of the pre-amplification steps described herein). In some embodiments, the pre-amplification step can, e.g., include: hybridization of one or more pairs (e.g., two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, one, two, three, four, five, six, seven, eight, nine, ten, eleven, twelve, thirteen, fourteen, fifteen, sixteen, seventeen, eighteen, nineteen, or twenty pairs) of a pre-amplification forward and reverse primer, where each pair of pre-amplification forward and reverse primers is designed to amplify 100 base pairs to 500 base pairs (e.g., between about 150 base pairs to 450 base pairs, between about 200 base pairs to about 400 base pairs, between about 200 base pairs to about 350 base pairs, or between about 250 base pairs and 300 base pairs) of genomic DNA that contains one of the one or more target SNPs (e.g., any of the exemplary SNPs described herein), where the pre-amplification forward and reverse primers in each of the one or more pairs of pre-amplification primers contains: (i) sequence of about 10 to about 30 contiguous nucleotides (e.g., about 13 to about 30 contiguous nucleotides, about 15 to about 30 contiguous nucleotides, about 17 to about 30 contiguous nucleotides, or about 17 to about 25 contiguous nucleotides) that is complementary to a sequence in the genomic DNA and (ii) a tag sequence of about 5 to about 25 contiguous nucleotides (e.g., between about 10 and 20 contiguous nucleotides, between about 5 and about 20 contiguous nucleotides, or between about 17 and about 25 contiguous nucleotides) that is not complementary to a sequence in the genomic DNA; and amplification of the genomic DNA using the one or more pairs of pre-amplification forward and reverse primers to generate 100 base pair to 500 base pair (e.g., 250 base pair to 300 base pair products). In some embodiments, the pre-amplification method further includes amplification of the 100 base pair to 500 base pair (e.g., 250 base pair to 300 base pair products) using a primer that comprises a sequence of about 5 to about 25 contiguous nucleotides (e.g., between about 10 and 20 contiguous nucleotides, between about 5 and about 20 contiguous nucleotides, or between about 17 and about 25 contiguous nucleotides) of the tag sequence or complementary to the tag sequence. In some embodiments, the tag sequence can include or be SEQ ID NO: 1. The at least two pairs of pre-amplification forward and reverse primers can be, e.g., selected from the group of: SEQ ID NO: 2 and SEQ ID NO: 3, respectively; SEQ ID NO: 4 and SEQ ID NO: 5, respectively; SEQ ID NO: 6 and SEQ ID NO: 7, respectively; SEQ ID NO: 8 and SEQ ID NO: 9, respectively; SEQ ID NO: 10 and SEQ ID NO: 11, respectively; SEQ ID NO: 12 and SEQ ID NO: 13, respectively; SEQ ID NO: 14 and SEQ ID NO: 15, respectively; SEQ ID NO: 16 and SEQ ID NO: 17, respectively; SEQ ID NO: 18 and SEQ ID NO: 19, respectively; SEQ ID NO: 20 and SEQ ID NO: 21, respectively; SEQ ID NO: 22 and SEQ ID NO: 23, respectively; SEQ ID NO: 24 and SEQ ID NO: 25, respectively; SEQ ID NO: 26 and SEQ ID NO: 27, respectively; SEQ ID NO: 28 and SEQ ID NO: 29, respectively; SEQ ID NO: 30 and SEQ ID NO: 31, respectively; and SEQ ID NO: 32 and SEQ ID NO: 33, respectively.

### Assays to Identify the Presence of One or More Saliva Proteins in Urine Sample

Some embodiments of any of the methods provided herein further include performing an assay to identify the presence of one or more saliva proteins (e.g., human statherin, human human alpha-amylase, and human lysozyme) in the urine sample.

Statherin is a unique phoshoprotein found in saliva. Human statherin is 62 amino acids in length. The human statherin protein sequence is shown below. A variety of antibodies that specifically bind to human statherin are commercially available (e.g., antibodies available from Santa Cruz Biotech, Abcam, and Acris).

Human Statherin Protein
(SEQ ID NO: 34)
MKFLVFAFILALMVSMIGADSSEEKFLRRIGRFGYGYGPYQPVPEQPLY
PQPYQPQYQQYTF

Human alpha-amylase is another protein that is present in saliva. Human alpha-amylase is 511 amino acids in length. The human alpha-amylase protein sequence is shown below. A variety of antibodies that specifically bind to human alpha-amylase are commercially available (e.g., antibodies available from BioVision, AbCam, Sigma-Aldrich, Novus Biologicals, and New England Biolabs).

Human Alpha-Amylase Protein
(SEQ ID NO: 35)
MKFFLLLFTIGFCWAQYSPNTQQGRTSIVHLFEWRWVDIALECERYLAP
KGFGGVQVSPPNENVAIYNPFRPWWERYQPVSYKLCTRSGNEDEFRNMV
TRCNNVGVRIYVDAVINHMCGNAVSAGTSSTCGSYFNPGSRDFPAVPYS
GWDFNDGKCKTGSGDIENYNDATQVRDCRLTGLLDLALEKDYVRSKIAE
YMNHLIDIGVAGFRLDASKHMWPGDIKAILDKLHNLNSNWFPAGSKPFI
YQEVIDLGGEPIKSSDYFGNGRVTEFKYGAKLGTVIRKWNGEKMSYLKN
WGEGWGFVPSDRALVFVDNHDNQRGHGAGGASILTFWDARLYKMAVGFM
LAHPYGFTRVMSSYRWPRQFQNGNDVNDWVGPPNNNGVIKEVTINPDTT
CGNDWVCEHRWRQIRNMVIFRNVVDGQPFTNWYDNGSNQVAFGRGNRGF
IVFNNDDWSFSLTLQTGLPAGTYCDVISGDKINGNCTGIKIYVSDDGKA
HFSISNSAEDPFIAIHAESKL

Human lysozyme is another protein that is present in saliva. Human lysozyme is 148 amino acids. The human lysozyme protein sequence is shown below. A variety of antibodies that specifically bind to human lysozyme are commercially available (e.g., antibodies available from AbCam, Thermo Scientific, Novus Biologicals, and AbD Serotec).

Human Lysozyme
(SEQ ID NO: 36)
MKALIVLGLVLLSVTVQGKVFERCELARTLKRLGMDGYRGISLANWNICL
AKWESGYNTRATNYNAGDRSTDYGIFQINSRYWCNDGKTPGAVNACHLSC
SALLQDNIADAVACAKRVVRDPQGIRAWVAWRNRCQNRDVRQYVQGCGV

A variety of antibody-based assays can be used to determine the presence of one or more of saliva proteins (e.g., statherin, alpha-amylase, and lysozyme) in the urine sample are also well known in the art. Non-limiting examples of antibody-based assays include enzyme-linked immunosorbent assays, immunoblotting, protein chip, beads (e.g., magnetic beads) that are coated with an antibody, immunoelectrophoresis, and immunoprecipitation. In some embodiments, any of the exemplary antibodies that bind specifically to one of statherin, alpha-amylase, or lysozyme can be used in any of the antibody-based assays described herein or known in the art to determine the presence or level of statherin, alpha-amylase, or lysozyme in a urine sample.

Additional assays for determining the presence or level of one or more saliva proteins (e.g., statherin, alpha-amylase, and lysozyme) in a urine sample are well known in the art and include without limitation: mass spectrometry, enzyme activity assays (e.g., using a detectable substrate or product), electrophoresis, and protein sequencing.

### Drugs and Drug Metabolites

Some of the methods described herein further include performing an assay to determine the level of one or more (e.g., two, three, four, five, six, or seven) drugs and/or the level one or more (e.g., two, three, four, five, six, or seven) drug metabolites (e.g., any of the exemplary drugs and/or drug metabolites described herein or known in the art) in a sample (e.g., a urine sample (e.g., a urine sample identified using any of the methods described herein as originating from the subject and/or identified using any of the methods described herein as not being adulterated) or a sample comprising blood, serum, hair, or plasma from a subject (e.g., a subject identified as providing a urine sample not originating from the subject (e.g., using any of the methods described herein) and/or a subject identified as providing an adulterated urine sample (e.g., using any of the methods described herein))).

Non-limiting examples of drugs and drug metabolites include: Δ9-tetrahydrocannabinol, Δ9-tetrahydrocannabino-11-oic acid, 11-hydroxy-Δ9-tetrahydrocannabinol, 11-nor-9-carboxy-Δ9-tetrahydrocannabinol, ethyl glucuronide, ethyl sulfate, morphine-3-glucuronide, morphine-6-glucu-ronide, amitriptyline, morphine 3,6-diglucuronide, morphine 3-ethereal sulfate, normorphine, cyclobenzaprine, norcodeine, codeine, normeperidine, norfentanyl, normorphine 6-glucoronide, 6-monoacetylmorphine, 6-monoacetylmorphine, 3-monoacetylmorphine, buprenorphine, morphine, clobazam, hydromorphone, hydrocodone, norhydrocodone, oxymorphone, normethadol, methadol, EDDP, EMDP, benzoylecgonine, ecgonine methyl ester, norcocaine, carisoprodol, p-hydroxycocaine, m-hydroxycocaine, p-hydroxybenzoylecgonine, m-hydroxybenzoylecgonine, methamphetamine, meperidine, meprobamate, amphetamine, MDMA, MDEA, MDA, 5-(glutathion-S-yl)-alpha-methyldopamine, 2,5-bis(glutathion-S-yl)-alpha-methyldopamine, free HMMA, DHMA sulfate, HMMA glucuronide, 7-aminoflunitrazepam, N-desmethylflunitrazepam, nitrazepam, N-desmethylclomipramine, N-desmethylcyclobenzaprine, doxepin, N-desmethylclobazam, desmethyldoxepin, 3-hydroxyflunitrazepam, gamma-hydroxybutyric acid, D-2-hydroxyglutaric acid, dehydronorketamine, maprotiline, imipramine, norketamine, 4-phenyl-4-(1-piperidinyl)cyclohexanol, dextrorphan, N-acetyl mescaline, ortriptyline, desipramine, 10-OH-nortriptyline, nortriptyline, tramadol, O-desmethyl-cis-tramadol, desmethyl-nortriptyline, 3-methylfentanyl, fentanyl, phenobarbital, amylobarbitone, 3′-hydroxyamylobarbitone, alpha-hydroxy alprazolam, zopiclone, zolpidem, 7-amino-clonazepam, 4-hydroxymidazolam, loprazolam, flurazepam, flurazepam, 7-aminoflunitrazepam, midazolam, 1-hydroxymidazolam, norbuprenorphine, bromazepam, primidone, alpha-hydroxyalprazolam, 3-hydroxyflunitrazepam, estralozam, pentazocine, alprazolam, lorazepam, clonazepam, triazolam, desalkylfurazepam, flunitrazepam, propoxyphene, protriptyline, ritalinic acid, lormetazepam, alpha-hydroxytriazolam, desmethylflunitrazepam, methadone, diazepam, dothiepin, nordiazepam, oxazepam, methylphenidate, mianserin, naloxone, N-desmethylmirtazapine, mirtazapine, N-desmethyltapentadol, tapentadol, N-desmethyltrimipramine, trimipramine, metagynine, 7-hydroxymitragynine, AM2201, HU-210, JWH-018, JWH-018 5-pentanoic acid metabolite, JWH-073, JWH-073 4-butanoic acid metabolite, JWH-073 N-(3-hydroxybutyl) metabolite, JWH-200, JWH-250, temazepam, marijuana, hashish, heroin, MPPP, ANPP, an opiate, cocaine, an amphetamine, phentermine, pregabalin, methamphetamine, a MDMA, flunitrazepam, GHB, ketamine, PCC, PCP, PEPAP, TCP, TCPy, Salvia divinorum, dextromethorphan, dextromorphan, LSD, mescaline, psilocybin, mephedrone, methylone, 3,4,-methylenedioxypyrovalerone (MDPV), phencyclidine, 17-alpha-methyl-1-testosterone, 4-Dihydrotestosterone (17beta-hydroxyandrostan-3-one), deltal-dihydrotestosterone (17beta-hydroxy-5alphaandrost-1-en-3-one), diazepam (Valium), zolpidem (Ambien, Ivadal, Stinoct, Stilnox), alprazolam (Xanax), alpha-ethyltryptamine, an anabolic steroid, an inhalant, acetaminophen, hydrocodone, noroxycodone, oxycodone, tricyclic antidepressants, barbituates, and benzodiazepines, and any precursors thereof.

A variety of urine drug assays and urine drug metabolite assays are commercially available. For example, urine drug metabolite assays can be purchased from American Screening Corp., Ameritox, Confirm Biosciences, Alibaba, Rapid Exams, DrugConfirm.

An assay to determine the level of one or more drugs and/or the level of one or more drug metabolites in a urine sample (e.g., any of the urine samples described herein from any subject described herein) can be performed at the same time, substantially the same time, or during an overlapping time period as one or more of: an assay to determine the mass distribution of a set of SNP amplification products in a urine sample (e.g., an assay to determine the mass distribution of a set of SNP amplification products using matrix-assisted laser desorption/ionization time-of-flight (MALDI-TOF) analysis) in the urine sample (e.g., using an aliquot of the same urine sample), an assay to determine the control mass distribution, an assay to determine the presence of one or more saliva proteins in the urine sample (e.g., using an aliquot of the same urine sample), and an assay to determine the genotype of at least one SNP in the isolated genomic DNA test sample or a control sample (e.g., using an aliquot of the same urine sample) is performed.

As is well known in the art, the determined level of the one or more drugs and/or the determined level of the one or more drug metabolites can be compared to reference values of the one or more drugs and/or the one or more drug metabolites (e.g., the level of the one or more drugs and/or the level of one or more drug metabolites in a subject that has not been administered a drug and/or an agent that is not metabolized into the one or more drug metabolites).

### Additional Exemplary Steps

In some embodiments, any of these methods further include recording the identification in step (h), the identification in step (j), and/or the identification in step (l) in the subject's medical record (e.g., a computer readable medium). In some embodiments, any of these methods further include notifying the subject's insurance provider, employer, or potential future employer of the identification in step (h), the identification in step (j), and/or the identification in step (l). In some embodiments, any of these methods further include notifying a pharmacist or a medical professional (e.g., any of the exemplary medical professionals described herein) of the identification in step (h), the identification in step (j), and/or the identification in step (l).

In some embodiments, any of these methods further include (i) selecting a subject having a urine sample identified in step (h) as not originating from the subject; and (j) obtaining an additional urine sample from the subject. In some embodiments, the additional urine sample is obtained through a witnessed urine test.

In some embodiments, any of these methods further include (i) selecting a subject having a urine sample identified in step (h) as not originating from the subject; (j) obtaining an additional urine sample (e.g., a witnessed urine sample) from the selected subject; and (k) performing an assay to determine the level of one or more drug metabolites in the urine sample from step (j).

In some embodiments, any of these methods further include (l) identifying a subject having an elevated level of one or more drugs and/or an elevated level of one or more drug metabolites in the additional urine sample as compared to a reference level of the one or more drugs and/or a reference level of one or more drug metabolites, wherein the drugs are an illegal or controlled substance and/or the drug metabolites are metabolites of an illegal or controlled substance; and (m) admitting the identified subject into a drug dependency program (e.g., a drug dependency program that includes administering to the admitted subject a drug replacement therapy), ceasing administration of the controlled substance to the identified subject, or reducing the dose and/or frequency of administration of the controlled substance to the identified subject. In some embodiments, step (m) can include administering a drug replacement therapy.

In some embodiments, any of these methods further include (i) selecting a subject having a urine sample identified in step (h) as not originating from the subject, (j) obtaining a sample comprising blood, serum, hair, skin, or plasma from the subject, and (k) performing an assay to determine the level of one or more drugs and/or the level of one or more drug metabolites in the sample from step (j) (e.g., blood, serum, hair, skin, or plasma).

In some embodiments, any of these methods further include (l) identifying a subject having an elevated level of one or more drugs and/or an elevated level of one or more drug metabolites in the sample from step (j) as compared to a reference level of the one or more drugs and/or a reference level of the one or more drug metabolites, where the drugs are an illegal or controlled substance and/or the drug metabolites are metabolites of an illegal or controlled substance; and (m) admitting the identified subject into a drug dependency program, ceasing administration of the controlled substance to the identified subject, or reducing the dose or frequency of administration of the controlled substance to the identified subject. In some embodiments, admitting the identified subject into a drug dependency program includes to the admitted subject a drug replacement therapy.

Some examples where the urine sample is identified in step (h) as originating from the subject can further include performing an assay to determine the level of one or more drug metabolites in the urine sample identified in step (h) as originating from the subject. Some examples further include identifying a subject having an elevated level of one or more drug metabolites in the urine sample identified in step (h) as originating from the subject, as compared to a reference level of the one or more drug metabolites, where the drug metabolites are metabolites of an illegal or controlled substance; and admitting the subject into the drug dependency program, ceasing administration of the controlled substance to the subject, or reducing the dose and/or frequency of administration of the controlled substance to the subject. In some examples, the drug dependency program includes administering to the subject a drug replacement therapy.

### Obtaining an Additional Urine Sample

Some embodiments of any of the methods described herein can further include identifying a urine sample as not originating from the subject and/or as being adulterated, and obtaining an additional urine sample from the subject. In some embodiments, the additional urine sample is obtained through a witnessed urine test.

Some embodiments of any of the methods described herein can further include identifying a urine sample as not originating from the subject and/or as being adulterated, and obtaining a sample including blood, serum, hair, or plasma from the subject.

### Kits

Also provided herein are kits that consist essentially of or consist of a set of two or more (e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 42 or more, 44 or more, 46 or more, 48 or more, 50 or more, 52 or more, 54 or more, 56 or more, 58 or more, 60 or more, 62 or more, 64 or more, 66 or more, 68 or more, 70 or more, 72 or more, 74 or more, 76 or more, 78 or more, 80 or more, 82 or more, 84 or more, 86 or more, 88 or more, 90 or more, 92 or more, 94 or more, 96 or more, 98 or more, or 100 or more) oligonucleotide probes, where each of the two or more oligonucleotide probes is capable of hybridizing (specifically hybridizing) to a sequence of genomic DNA that is one, two, three, four, five, six, seven, eight, nine, or ten nucleotides upstream of a different target SNP. Any of the sets of two or more oligonucleotide probes described herein can be included in the kits. Any of the aspects of the set of two or more oligonucleotide probes described herein can be utilized in the set of two or more oligonucleotide probes included in any of the kits described herein.

In some embodiments, the kit can further include chain-terminating nucleotide(s) (e.g., any of the chain-terminating nucleotide(s) described herein).

In some embodiments, the kit can further include two or more (e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 42 or more, 44 or more, 46 or more, 48 or more, 50 or more, 52 or more, 54 or more, 56 or more, 58 or more, 60 or more, 62 or more, 64 or more, 66 or more, 68 or more, 70 or more, 72 or more, 74 or more, 76 or more, 78 or more, 80 or more, 82 or more, 84 or more, 86 or more, 88 or more, 90 or more, 92 or more, 94 or more, 96 or more, 98 or more, or 100 or more) pairs of a pre-amplification forward and reverse primer, where each pair of forward and reverse primers is designed to amplify 100 base pairs to 500 base pairs (e.g., about 100 base pairs to about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, about 220 base pairs, about 200 base pairs, about 180 base pairs, about 160 base pairs, about 140 base pairs, or about 120 base pairs (inclusive); about 120 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, about 220 base pairs, about 200 base pairs, about 180 base pairs, about 160 base pairs, or about 140 base pairs (inclusive); about 140 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, about 220 base pairs, about 200 base pairs, about 180 base pairs, or about 160 base pairs (inclusive); about 160 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, about 220 base pairs, about 200 base pairs, or about 180 base pairs (inclusive); about 180 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, about 220 base pairs, or about 200 base pairs (inclusive); about 200 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, about 240 base pairs, or about 220 base pairs (inclusive); about 220 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, about 260 base pairs, or about 240 base pairs (inclusive); about 240 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, about 280 base pairs, or about 260 base pairs (inclusive); about 260 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, about 300 base pairs, or about 280 base pairs (inclusive); about 280 base pairs to about 500 base pairs, about 480 base pairs, about 460 base pairs, about 440 base pairs, about 420 base pairs, about 400 base pairs, about 380 base pairs, about 360 base pairs, about 340 base pairs, about 320 base pairs, or about 300 base pairs (inclusive); about 300 base pairs to about 500 base pairs, about 480 base pairs, about 460 base pairs, about 440 base pairs, about 420 base pairs, about 400 base pairs, about 380 base pairs, about 360 base pairs, about 340 base pairs, or about 320 base pairs (inclusive); about 320 base pairs to about 500 base pairs, about 480 base pairs, about 460 base pairs, about 440 base pairs, about 420 base pairs, about 400 base pairs, about 380 base pairs, about 360 base pairs, or about 340 base pairs (inclusive); about 340 base pairs to about 500 base pairs, about 480 base pairs, about 460 base pairs, about 440 base pairs, about 420 base pairs, about 400 base pairs, about 380 base pairs, or about 360 base pairs (inclusive); about 360 base pairs to about 500 base pairs, about 480 base pairs, about 460 base pairs, about 440 base pairs, about 420 base pairs, about 400 base pairs, or about 380 base pairs (inclusive); about 380 base pairs to about 500 base pairs, about 480 base pairs, about 460 base pairs, about 440 base pairs, about 420 base pairs, or about 400 base pairs (inclusive); about 400 base pairs to about 500 base pairs, about 480 base pairs, about 460 base pairs, about 440 base pairs, or about 420 base pairs (inclusive); about 420 base pairs to about 500 base pairs, about 480 base pairs, about 460 base pairs, or about 440 base pairs (inclusive); about 440 base pairs to about 500 base pairs, about 480 base pairs, or about 460 base pairs (inclusive); about 460 base pairs to about 500 base pairs or about 480 base pairs (inclusive); or about 480 base pairs to about 500 base pairs (inclusive)) of genomic DNA that contains at least one target SNP, where the pre-amplification forward and reverse primers in each of the two or more pairs contains (i) a sequence of about 10 contiguous nucleotides to about 30 contiguous nucleotides (e.g., about 10 contiguous nucleotides to about 28 contiguous nucleotides, about 26 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, or about 12 contiguous nucleotides (inclusive); about 12 contiguous nucleotides to about 30 contiguous nucleotides, about 28 contiguous nucleotides, about 26 contiguous nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 30 contiguous nucleotides, about 28 contiguous nucleotides, about 26 contiguous nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 30 contiguous nucleotides, about 28 contiguous nucleotides, about 26 contiguous nucleotides, about 24 contiguous nucleotides, about 22 contiguous nucleotides, about 20 contiguous nucleotides, or about 18 contiguous nucleotides (inclusive); about 18 contiguous nucleotides to about 30 contiguous nucleotides, about 28 contiguous nucleotides, about 26 contiguous nucleotides, about 24 contiguous nucleotides, about 22 contiguous nucleotides, or about 20 contiguous nucleotides (inclusive); about 20 contiguous nucleotides to about 30 contiguous nucleotides, about 28 contiguous nucleotides, about 26 contiguous nucleotides, about 24 contiguous nucleotides, or about 22 contiguous nucleotides (inclusive); about 22 contiguous nucleotides to about 30 contiguous nucleotides, about 28 contiguous nucleotides, about 26 contiguous nucleotides, or about 24 contiguous nucleotides (inclusive); about 24 contiguous nucleotides to about 30 contiguous nucleotides, about 28 contiguous nucleotides, or about 26 contiguous nucleotides (inclusive); about 26 contiguous nucleotides to about 30 contiguous nucleotides or about 28 contiguous nucleotides (inclusive); or about 28 contiguous nucleotides to about 30 contiguous nucleotides (inclusive)) that is complementary to a sequence in the genomic DNA and (ii) a tag sequence of about 5 contiguous nucleotides to about 25 contiguous nucleotides (e.g., about 5 contiguous nucleotides to about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 6 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 8 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, or about 10 contiguous nucleotides (inclusive); about 12 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 25 contiguous nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 25 contiguous nucleotides, about 24 contiguous nucleotides, about 22 contiguous nucleotides, about 20 contiguous nucleotides, or about 18 contiguous nucleotides (inclusive); about 18 contiguous nucleotides to about 25 contiguous nucleotides, about 24 contiguous nucleotides, about 22 contiguous nucleotides, or about 20 contiguous nucleotides (inclusive); about 20 contiguous nucleotides to about 25 contiguous nucleotides, about 24 contiguous nucleotides, or about 22 contiguous nucleotides (inclusive); about 22 contiguous nucleotides to about 25 contiguous nucleotides, or about 24 contiguous nucleotides (inclusive); or about 23 contiguous nucleotides to about 25 contiguous nucleotides (inclusive)) that is not complementary to a sequence in the genomic DNA; and a primer that comprises a sequence of about 5 contiguous nucleotides to about 25 contiguous nucleotides (e.g., about 5 contiguous nucleotides to about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 6 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, about 10 contiguous nucleotides, or about 8 contiguous nucleotides (inclusive); about 8 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, about 14 contiguous nucleotides, about 12 contiguous nucleotides, or about 10 contiguous nucleotides (inclusive); about 12 contiguous nucleotides to about 25 contiguous nucleotides, about 24 contiguous nucleotides, about 22 contiguous nucleotides, about 20 contiguous nucleotides, about 18 contiguous nucleotides, about 16 contiguous nucleotides, or about 14 contiguous nucleotides (inclusive); about 14 contiguous nucleotides to about 25 contiguous nucleotides, about 24 contiguous nucleotides, about 22 contiguous nucleotides, about 20 contiguous nucleotides, about 18 contiguous nucleotides, or about 16 contiguous nucleotides (inclusive); about 16 contiguous nucleotides to about 25 contiguous nucleotides, about 24 contiguous nucleotides, about 22 contiguous nucleotides, about 20 contiguous nucleotides, or about 18 contiguous nucleotides (inclusive); about 18 contiguous nucleotides to about 25 contiguous nucleotides, about 24 contiguous nucleotides, about 22 contiguous nucleotides, or about 20 contiguous nucleotides (inclusive); about 20 contiguous nucleotides to about 25 contiguous nucleotides, about 24 contiguous nucleotides, or about 22 contiguous nucleotides (inclusive); about 22 contiguous nucleotides to about 25 contiguous nucleotides, or about 24 contiguous nucleotides (inclusive); or about 23 contiguous nucleotides to about 25 contiguous nucleotides (inclusive)) of the tag sequence or complementary to the tag sequence. Any of the aspects of the two or more pairs of pre-amplification forward and reverse primers described herein can be included in the two or more pairs of pre-amplification forward and reverse primers included in any of the kits described herein.

In some embodiments, the kit can further include an enzyme-linked immunosorbent assay (ELISA) for detection of one or more saliva proteins (e.g., one of more of human statherin, human alpha-amylase, or human lysozyme), an antibody that binds specifically to a saliva protein (e.g., human statherin, human alpha-amylase, or human lysozyme) and/or a labeled substrate for detection of the activity (e.g., binding activity or enzymatic activity) of one or more saliva proteins (e.g., human statherin, human alpha-amylase, or human lysozyme).

The invention is further described in the following example, which does not limit the scope of the invention described in the claims.

### Example 1. DNA Verified Sample Authenticity for Urine Drug Testing of Chronic Pain Patients

Urine Drug Testing (UDT) is used to monitor patient compliance with a prescribed treatment plan. Providers rely on the results of UDT to make critical care decisions. Modern UDT can provide a rich and complete picture of the substances a is person has been taking, but is not foolproof.

Experiments were performed to determine which patients are likely to attempt to cheat a urine drug test and how are these patients trying to cheat the urine drug test. Patients may try to cheat a urine drug test by substituting urine from another individual. Patients may also attempt to cheat a urine drug test by using synthetic urine Patients may also try to cheat a urine drug test by “Pill Scraping,” an adulteration technique in which medication is added directly to urine. Experiments described herein were performed to provide a detailed analysis of anomalous test results among a population of 10,000 urine specimens. The experiments described below combined the results of validity, toxicology, synthetic urine screening, and DNA authentication (as performed generally using the methods described herein).

### Methods

Genetic authentication of urine was performed by matching the genotype of multiple Single Nucleotide Polymorphism (SNP) markers between DNA samples collected from the urine sample and from the buccal cell sample of its claimed donor (performed generally using the methods described herein). Urine validity test measures were supplemented with a method for identification of synthetic urine. Toxicology data were obtained through quantitative LC-MS/MS analysis. Each urine sample was scanned using a proprietary method analyzing compound constituency to determine whether it was real or synthetic.

### Results

### Prevalence of Adulteration & Substitution

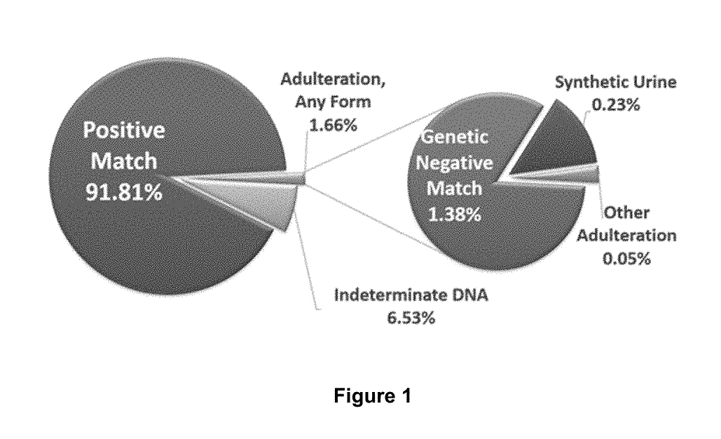

DNA-authenticated urine was observed in 91.9% of specimens (N=10,000). Indeterminate DNA results due to low DNA quality or degradation were 6.5%. Anomalous genetic results accounted for nearly 1.4% of specimens submitted for authentication. This includes genetic negative matches as well as the lack of DNA as in the case of synthetic urine. While traditional validity testing can sometimes identify dilution with water, it fails to observe the most common forms of adulteration and substitution. Of specimens presenting evidence of substitution of urine, 98% of these pass all traditional validity tests (FIG. 1).

### Inadequacy of Current Validity Tests

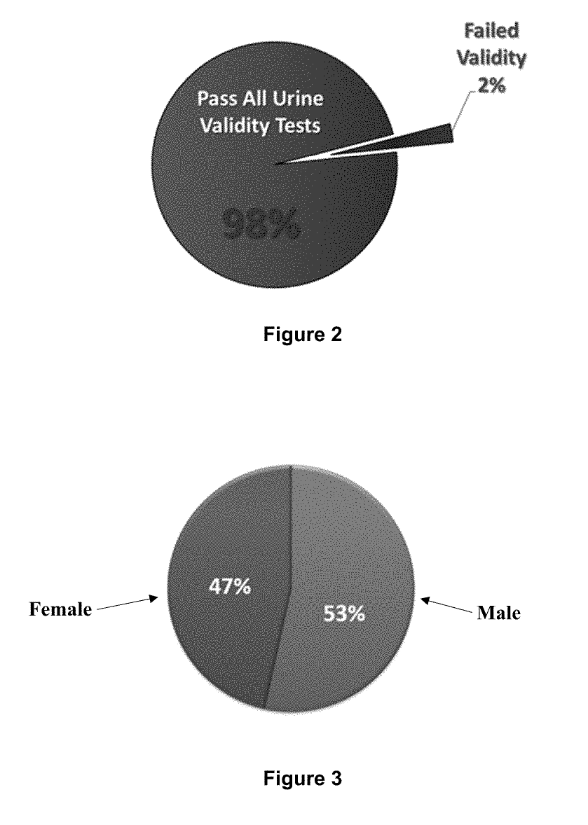

Traditional validity can identify dilution, but without genetic verification it is not always clear if the result is due to adulteration or high levels of patient hydration. Of the identified cases of genetic mismatch and synthetic urine, 98% pass all validity measures and 100% would be reported as valid urine specimens using traditional testing procedures (FIG. 2).

### Age and Sex in Cases of Genetic Mismatch

DNA analysis of urine is able to determine the sex of the donor. Approximately 20% of genetic mismatch cases indicate urine originating from a member of the opposite sex. In 2% of the cases, the DNA in urine matched the DNA of another person at the same clinic. Men and women are similarly likely to substitute urine with 47% of negative-match cases being female. In cases of genetic mismatch the most common prescriptions are for buprenorphine/naloxone combinations (FIG. 3). Together, these medicines are prescribed in approximately 40% of adulteration cases. In substituted urine, the most common substance that is observed is cotinine (approximately 40% of cases), indicating a correlation between nicotine addiction and drug seeking behavior.

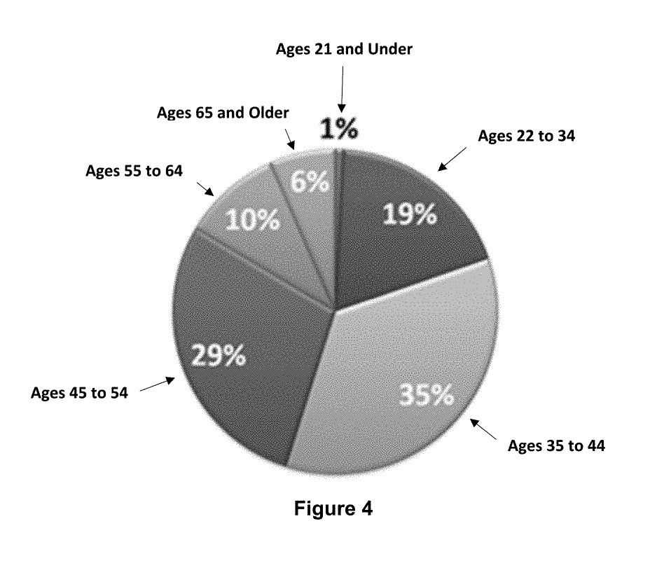

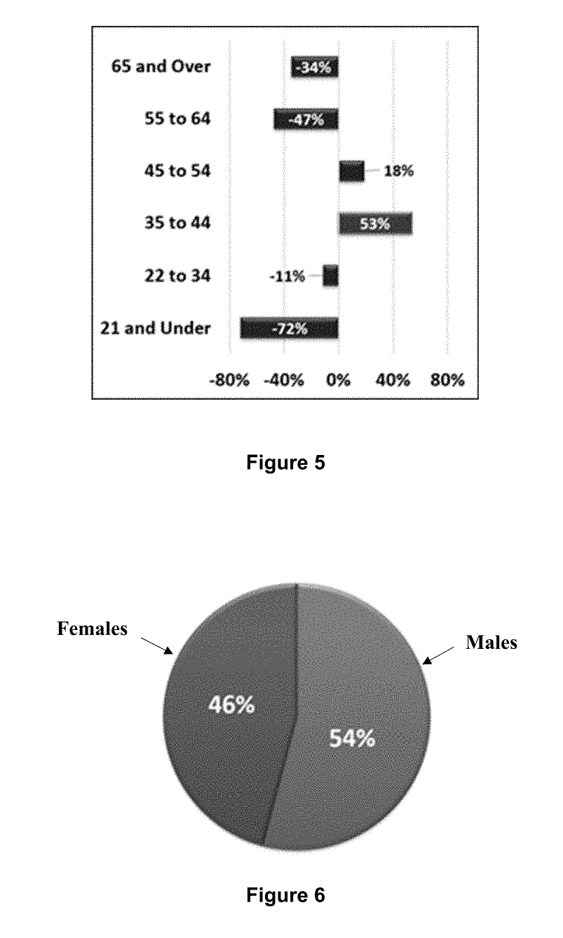

Cheating by urine substitution occurs among all age groups including the elderly (>65). It is most prevalent in people between the ages of 35 and 54 (FIG. 4). When the frequency of substitute urine in each age group (as measured by genetic negative match) is normalized and centered about the mean frequency, it becomes that apparent that individuals older than 55 are the least likely to cheat, while ages 35 to 55 are 53% more likely to cheat than the average (FIG. 5).

### Synthetic Urine Use

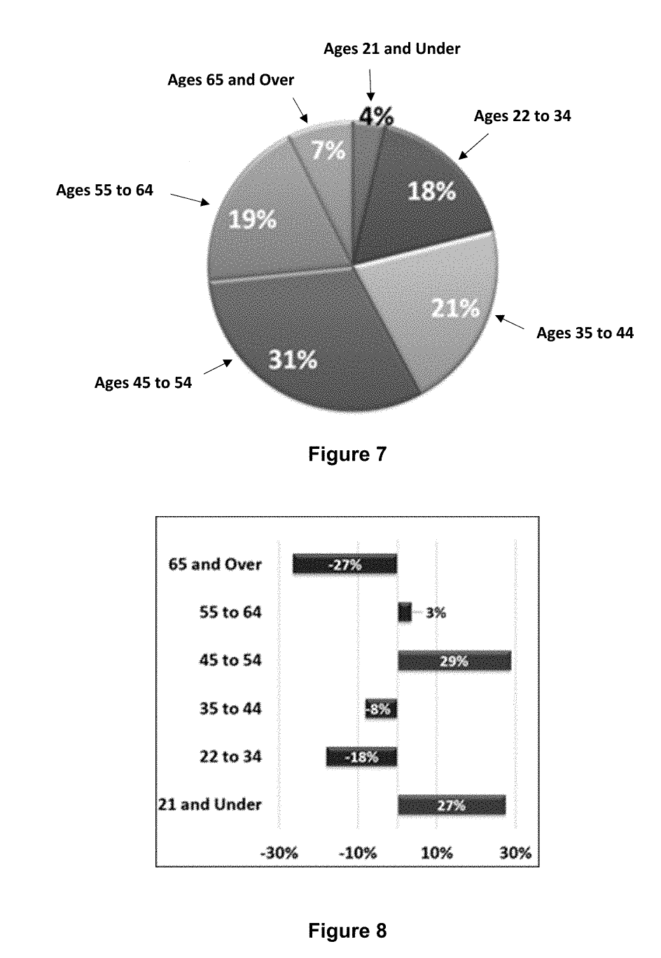

Over 30 varieties of commercially available synthetic urine or urine substitutes available from online vendors were evaluated. Thirty-one of the 33 varieties pass all of the traditional validity measures' specific gravity, creatinine content, pH, and oxidant adulteration. The other two showed low creatinine but were valid by other measures and would have been reported as acceptable urine using standard procedures. Synthetic urine detection was performed to detect synthetic urine use in a population of 49,494 urine specimens using the methods generally described herein. Use of synthetic urine is favored slightly by males in a similar trend to that observed for the substitution of another individual's urine (FIG. 6). Use of synthetic urine is favored by persons age 45 to 54 and by those under 21 by approximately 30% compared with the population average (FIGS. 7 and 8).

### CONCLUSIONS

These data indicate that traditional validity measures for urine toxicology testing fail to identify the vast majority of cases of subversion of urine toxicology tests. As an arbitrary subset of total samples were submitted for genetic authentication, the true rate of urine substitution could differ from the discovered 1.4% Interestingly, the nature of genetic authentication may bias this otherwise undeterminable number by deterring urine substitution in would-be cases of UDT fraud. It is posited that the rate of 1.4% represents a lower bound for the true rate of adulteration and substitution in pain management settings.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of matching a urine sample to a subject comprising:
(a) providing a urine sample from a subject;
(b) enriching the urine sample for mammalian cells, if present;
(c) isolating any genomic DNA from the enriched sample of step (b) to form an isolated genomic DNA test sample;
(d) contacting a set of two or more oligonucleotide probes to the genomic DNA in the isolated genomic DNA test sample, wherein each of the two or more oligonucleotide probes hybridizes to a sequence of genomic DNA that is one nucleotide upstream of a different target single nucleotide polymorphism (SNP);
(e) elongating the two or more oligonucleotide probes hybridized to the genomic DNA by one nucleotide using chain-terminating nucleotide(s) to generate a set of two or more SNP amplification products;
(f) determining the mass distribution of the set of SNP amplification products;
(g) comparing the determined mass distribution of the set of SNP amplification products to a control mass distribution; and
(h) identifying a urine sample having a mass distribution that is the same as the control mass distribution as originating from the subject; or
identifying a urine sample having a mass distribution that is not the same as the control mass distribution as not originating from the subject.

2. The method of claim 1, wherein the chain-terminating nucleotide(s) is/are dideoxynucleotide(s).

3. The method of claim 1, wherein the mass distribution of the set of SNP amplification products in step (f) is determined using matrix-assisted laser desorption/ionization time-of-flight (MALDI-TOF) analysis.

4. The method of claim 1, wherein each of the two or more oligonucleotide probes hybridizes to a sequence of genomic DNA that is one nucleotide upstream of a target SNP having a minor allele frequency of >0.4.

5. The method of claim 4, wherein the set of oligonucleotide probes includes at least four oligonucleotide probes.

6.-17. (canceled)

18. The method of claim 1, wherein the subject is a genetic male and at least one target SNP is located on a Y chromosome.

19. The method of claim 5, wherein the target SNPs include at least one target SNP from at least three different chromosomes.

20. (canceled)

21. The method of claim 1, further comprising between steps (c) and (d) a step of:
performing a pre-amplification step.

22. The method of claim 21, wherein the pre-amplification step includes:
hybridization of two or more pairs of a pre-amplification forward and reverse primer, wherein each pair of pre-amplification forward and reverse primers is designed to amplify 250 to 300 nucleotides of genomic DNA that contains one of the different target SNPs, where the pre-amplification forward and reverse primer in each of the pairs of pre-amplification primers contains (i) a sequence of about 17 to 25 contiguous nucleotides that is complementary to a sequence in the genomic DNA and (ii) a tag sequence of about 17 to about 25 contiguous nucleotides that is not complementary to a sequence in the genomic DNA; and
amplification of the genomic DNA using the two or more pairs of pre-amplification forward and reverse primers to generate 250 to 300 nucleotide amplification product(s).

23. The method of claim 22, wherein the pre-amplification step further comprises amplification of the 250 to 300 nucleotide amplification product(s) using a primer that comprises a sequence of about 17 to 25 contiguous nucleotides complementary to the tag sequence.

24. The method of claim 1, wherein the control mass distribution is determined from a buccal cell sample obtained from the subject.

25. (canceled)

26. The method of claim 24, further comprising determining the mass distribution of the buccal cell sample obtained from the subject.

27. The method of claim 1, wherein the subject is a human.

28. The method of claim 1, further comprising:
(i) performing an assay to identify the presence of one or more of statherin, alpha-amylase, and lysozyme in the urine sample; and
(j) identifying a urine sample having a mass distribution that is the same as the control mass distribution and a detectable level of one or more of statherin, alpha-amylase, and lysozyme as being adulterated.

29. (canceled)

30. (canceled)

31. The method of claim 1, further comprising:
recording the identification in step (h) in the subject's medical record;
notifying the subject's insurance provider, employer, or potential future employer of the identification in step (h); or
notifying a pharmacist or a medical professional of the identification in step (h).

32.-34. (canceled)

35. The method of claim 1, further comprising:
(i) selecting a subject having a urine sample identified in step (h) as not originating from the subject;
(j) obtaining an additional urine sample from the selected subject; and
(k) performing an assay to determine the level of one or more drug metabolites in the additional urine sample.

36. (canceled)

37. (canceled)

38. The method of claim 35, further comprising:
(l) identifying a subject having an elevated level of one or more drug metabolites in the additional urine sample as compared to a reference level of the one or more drug metabolites, wherein the drug metabolites are metabolites of an illegal or controlled substance; and
(m) admitting the subject into the drug dependency program, ceasing administration of the controlled substance to the subject, or reducing the dose and/or frequency of administration of the controlled substance to the subject.

39. (canceled)

40. The method of claim 1, further comprising:
(i) selecting a subject having a urine sample identified in step (h) as not originating from the subject;
(j) obtaining a sample comprising blood, serum, hair, or plasma from the subject; and
(k) performing an assay to determine the level of one or more drug metabolites in the sample from step (j).

41. The method of claim 40, further comprising:
(l) identifying a subject having an elevated level of one or more drug metabolites in the sample from step (j) as compared to a reference level of the one or more drug metabolites in the sample from step (j) as compared to a reference level of the one or more drug metabolites, wherein the drug metabolites are metabolites of an illegal or controlled substance; and
(m) admitting the subject into a drug dependency program, ceasing administration of the controlled substance to the subject, or reducing the dose or frequency of administration of the controlled substance to the subject.

42. (canceled)

43. The method of claim 1, wherein the urine sample is identified in step (h) as originating from the subject, and the method further comprises performing an assay to determine the level of one or more drug metabolites in the urine sample identified in step (h) as originating from the subject.

44. (canceled)

45. The method of claim 43, further comprising:
identifying a subject having an elevated level of one or more drug metabolites in the urine sample identified in step (h) as originating from the subject, as compared to a reference level of the one or more drug metabolites, wherein the drug metabolites are metabolites of an illegal or controlled substance; and
admitting the subject into the drug dependency program, ceasing administration of the controlled substance to the subject, or reducing the dose and/or frequency of administration of the controlled substance to the subject.

46. (canceled)

47. The method of claim 1, wherein the subject has not been diagnosed as having an illegal or controlled substance addiction.

48. The method of claim 1, wherein:
the subject has been identified as having an illegal or controlled substance addiction;
the subject is being treated on an outpatient basis for an illegal or controlled substance addiction; or
the subject is entering into or participating in a medication monitoring program involving a controlled substance.

49. (canceled)

50. (canceled)
